(19) United States
(12) Patent Application Publication
(10) Pub. No.: US 2003/0134310 A1
(43) Pub. Date: Jul. 17, 2003

(54) Cellular kinase targets and inhibitors, and methods for their use

(76) Inventor: Thomas P. Cujec, Melrose, MA (US)

Correspondence Address: CLARK & ELBING LLP, 101 FEDERAL STREET, BOSTON, MA, 02110, US

(21) Appl. No.: 10/293,086

(22) Filed: Nov. 13, 2002

Related U.S. Application Data
(60) Provisional application No. 60/337,990, filed on Nov. 13, 2001.

(51) Int. Cl. C12Q 1/68; C07H 21/04; C12N 9/12; C12P 21/02; C12N 5/06

(52) U.S. Cl. 435/6; 435/69.1; 435/194; 435/320.1; 435/325; 536/23.2

(57) ABSTRACT

Described herein are polypeptides that are phosphorylation targets of a kinase, methods for identifying compounds that decrease the phosphorylation activity of a kinase, methods and reagents for inhibiting kinase activity, and methods of phosphorylating proteins. These methods are also particularly described with respect to the abl kinase.

### CROSS REFERENCE TO RELATED APPLICATIONS

This application claims the benefit of the filing date of U.S. provisional application, U.S. S. No. 60/337,990, filed Nov. 13, 2001, hereby incorporated by reference.

### BACKGROUND OF THE INVENTION

In general, the present invention relates to proteins that are phosphorylation targets of kinases (such as abl tyrosine kinases) and methods for identifying compounds that modulate, for example, inhibit phosphorylation by such kinases. In addition, the present invention relates to kinase inhibitors, for example, polypeptides that are identified by these methods.

Phosphorylation of proteins on their serine, threonine, and tyrosine residues is one of the most commonly occurring post-translational modifications in eukaryotic cells. Cellular phosphorylation cascades allow for the amplification of extra-cellular signals following changes in environmental conditions via the ability of phosphorylated activators to modulate the expression of numerous genes. Because these reactions are rapidly reversible, they are important for the regulation of many cellular functions including signal transduction, cell division, and proliferation. The specificity of these interactions is determined by a number of parameters, including cellular co-localization of the enzyme and its substrate, secondary interactions between the enzyme and the substrate, as well as the substrate specificity of the kinase catalytic domain (Acuto, Annu. Rev. Immunol. 18:165-184, 2000; Hunter, Cell 100:113-127, 2000; Pawson et al., Science 278:2075-2080, 1997). Identification of kinase substrate sequences is a critical step towards understanding the biology of these kinases, and provides an important venue for the development of kinase inhibitory reagents.

Numerous in vivo and in vitro protein display technologies have been developed in an attempt to decipher the myriad of biological pathways which occur in a cell (Mendelsohn et al., Science 284:1948-1950, 1999; Phizicky et al., Microbiol. Rev. 59:94-123, 1995). Strategies involving in vivo display of protein domains generally involve making proteomic libraries derived from cellular mRNA and then selecting for desired interactions either within the organism itself (yeast two-hybrid and its derivatives) (Brent et al., Annu. Rev. Genet 31:663-704, 1997; Fields et al., Nature 340:245-246, 1989; Vidal et al, Nucleic Acids Res. 27:919-929, 1999), or following cell surface expression (phage and E. coli display) (Hu et al., Methods 20:80-94, 2000; Johnsson et al., Curr. Top. Microbiol. Immunol. 243:87-105, 1999; Smith et al., Chem. Rev. 97:391-410, 1997; Wittrup, Trends Biotechnol. 17:423-424, 1999). Although these systems are capable of detecting protein-protein interactions, their ability to identify proteins targeted for post-translational modification has not been demonstrated. Numerous in vitro protein display systems have also been developed (Schaffitzel et al., J. Immunol. Methods 231:119-135, 1999; Shusta et al., Curr. Opin. Biotechnol. 10:117-122, 1999), however in most cases the absence of a robust linkage between genotype and phenotype limits their applicability in studying enzymatic modifications. Although methodologies for the identification of kinase substrates has generally lagged behind those aimed at elucidating protein-protein interactions, several approaches have been employed. These include phosphorylation of proteins immobilized on a nitrocellulose filter following induction of a phage expression library (Obata et al., J. Biol. Chem. 275:36108-36115, 2000), mutation of the kinase's ATP-binding site allowing it to accept N⁶-modified ATP analogues which can be used as substrate tags (Habelhah et al., J. Biol. Chem. 19:19, 2001), and the selection of phosphorylated targets from synthetic peptide libraries (Songyang et al., Curr. Biol. 4:973-982, 1994). In this latter approach, a random peptide library is phosphorylated in vitro by the kinase of interest, the substrates are recovered on a metal-chelating column and the mixture of proteins is sequenced.

### SUMMARY OF THE INVENTION

The present invention provides cellular targets of kinases, such as the abl kinase, identified by an improved technique. The non-receptor tyrosine kinase v-abl is encoded by the Abelson murine leukemia virus and is a potent oncogene in mice. The closely related human proto-oncogene c-abl contains the src-homology (SH) regions 1, 2, and 3, which confer phosphorylation activity (SH1), ability to bind phosphorylated tyrosines (SH2), and kinase inhibitory functions (SH3) on the protein (Rosenberg, Adv. Virus Res. 35:39-81, 1988; Zou et al., J. Biol. Chem. 274:18141-18144, 1999). A reciprocal translocation between c-abl (chromosome 9) and the bcr gene (chromosome 22) results in the potent oncogene BCR-abl, which is implicated in the pathogenesis of 95% of chronic myelogenous leukemias and 10% of acute lymphocytic leukemias (Cortes et al., Am. J. Med 100:555-570, 1996; Gishizky, Cytokines Mol. Ther. 2:251-261, 1996). Although numerous putative substrates of the abl kinases have been identified, including those involved in cell architecture, transcription, and many signal transduction pathways, the mechanism of action of these kinases remains unclear (Zou et al., J. Biol. Chem. 274:18141-18144, 1999; Laneuville, Semin. Immunol. 7:255-266, 1995; Pendergast, Curr. Opin. Cell. Biol. 8:174-181, 1996). A minimal v-abl consensus sequence (I/V/L-Y-Xₙ-P/F where n=2 or 3) has been previously identified from a peptide library randomized at eight amino acids, but the effects of larger sequence motifs on substrate selection is unknown (Songyang et al., Nature 373:536-539, 1995).

To identify optimal substrate sequences of the potent v-abl oncogene, we applied a selection based technology to a randomized peptide library. In addition, we identified cellular targets of v-abl from a proteomic mRNA-protein library derived from human bone marrow cells. The approach we describe here offers a general strategy for identifying novel substrates of kinases, such as tyrosine and serine/threonine kinases, and facilitates the rapid identification of lead candidates for kinase inhibitors.

Accordingly, in a first aspect, the invention features a substantially pure Shg polypeptide. In one embodiment, the substantially pure Shg polypeptide comprises the amino acid sequence of SEQ ID NO: 73.

In a related aspect, the invention features an isolated nucleic acid molecule encoding a Shg polypeptide. In one embodiment, the isolated nucleic acid molecule includes the nucleotide sequence of SEQ ID NO: 74. In other related aspects, the invention features vectors and cells containing a Shg nucleic acid, preferably positioned for expression of the Shg polypeptide.

In a second aspect, the invention features a substantially pure polypeptide that is phosphorylated by an abl kinase, where the polypeptide includes the sequence of any of SEQ ID NOS: 49-51, 127, 129, 131, 133, 135, 137, and 139.

In a related aspect, the invention features an isolated nucleic acid encoding a polypeptide that is phosphorylated by an abl kinase, where the polypeptide includes the sequence of any of SEQ ID NOS: 49-51, 127, 129, 131, 133, 135, 137, and 139. In one embodiment, the isolated nucleic acid sequence includes the sequence of any of SEQ ID NOS: 118-120, 128, 130, 132, 134, 136, 138 and 140.

In a third aspect, the invention features a substantially pure polypeptide that inhibits the activity of an abl kinase, where the polypeptide contains the sequence of any of SEQ ID NOS: 76-79, 81, and 83-85. In a related aspect, the invention features an isolated nucleic acid molecule encoding a polypeptide that inhibits the activity of an abl kinase.

In another related aspect, the invention features a method of inhibiting abl kinase activity, involving contacting the abl kinase with a polypeptide containing any of SEQ ID NOS: 76-79, 81, and 83-85. This method may be carried out in a cell. The cell may be in vivo or ex vivo.

In a fourth aspect, the invention features a method for identifying a protein phosphorylated by a kinase, and its coding sequence, the method involving the steps of: (a) contacting a population of nucleic acid-protein fusions with a kinase under conditions that allow phosphorylation of the protein portion of the nucleic acid-protein fusion by the kinase; (b) separating phosphorylated nucleic acid-protein fusions from nonphosphorylated nucleic acid-protein fusions; (c) amplifying the nucleic acid portions of the phosphorylated nucleic acid-protein fusions; and (d) repeating steps (a)-(c) one or more times, using the amplified nucleic acid of step (c) to generate an enriched population of nucleic acid-protein fusions for use in step (a), thereby identifying a protein phosphorylated by a kinase, and its coding sequence.

In preferred embodiments, in step (b), the phosphorylated nucleic acid-protein fusions are separated by immunoprecipitation (for example, using a phospho-specific antibody); the kinase is a tyrosine kinase (for example, an abl kinase) or the kinase is a serine or threonine kinase; and the nucleic acids used to generate the population of nucleic acid-protein fusions of step (a) are cellular mRNA, or are synthetic oligonucleotides or nucleic acid fragments.

In a related aspect, the invention features a method for identifying on a nucleic acid microarray a coding sequence for a protein phosphorylated by a kinase involving the above method steps (a)-(d), followed by the further steps of: (e) detectably labeling cDNA complementary to the nucleic acid portions of the enriched population of nucleic acid-protein fusions of step (d); (f) hybridizing the detectably labeled nucleic acids to a nucleic acid microarray; and (g) comparing the intensity of label associated with one or more microarray species to the intensity of label associated with said one or more microarray species when alternatively hybridized to labeled cDNA complementary to the nucleic acid portions of the unenriched library of step (a), whereby increased label in association with a microarray species hybridized to the cDNA from the enriched population of nucleic acid-protein fusions compared to the label when hybridized to cDNA from the unenriched library identifies that species as a nucleic acid that encodes a protein phosphorylated by the kinase.

In a fifth aspect, the invention features methods for identifying kinase inhibitors. The first method involves: (a) contacting a population of nucleic acid-protein fusions with a kinase under conditions that allow the nucleic acid-protein fusions to bind to the kinase; (b) separating kinase-bound nucleic acid-protein fusions from free nucleic acid-protein fusions; (c) amplifying the nucleic acid portions of the bound nucleic acid-protein fusions; (d) repeating steps (a)-(c) one or more times, using the amplified nucleic acids of step (c) to generate an enriched population of nucleic acid-protein fusions for use in step (a), thereby identifying one or more nucleic acid-protein fusions that bind the kinase; (e) separately contacting one or more kinase-binding nucleic acid-protein fusions, or the protein portions thereof, identified in step (d) with the kinase and its substrate under conditions that allow phosphorylation of the substrate by the kinase; and (f) determining the phosphorylation level of the substrate, a decreased level of phosphorylation of the substrate in the presence of the kinase-binding nucleic acid-protein fusion, or protein portion thereof, relative to the phosphorylation level of the substrate in the absence of the kinase-binding nucleic acid-protein fusion, or protein portion thereof, identifying the protein portion of the kinase-binding nucleic acid-protein fusion as a kinase inhibitor.

In preferred embodiments, the nucleic acid used to generate the population of nucleic acid-protein fusions of step (a) is cellular mRNA; and the kinase is a tyrosine kinase or a serine or threonine kinase.

In another method, the invention features a method for identifying a compound that decreases the phosphorylation activity of a kinase, involving providing a nucleic acid-protein fusion molecule, where the protein portion of the nucleic acid-protein fusion molecule can be phosphorylated by the kinase; contacting the nucleic acid-protein fusion molecule with the kinase and a candidate compound under conditions that allow phosphorylation of the protein portion of the nucleic acid-protein fusion molecule by the kinase; and determining the phosphorylation level of the nucleic acid-protein fusion molecule. A decreased level of phosphorylation of the fusion molecule in the presence of the candidate compound, relative to the phosphorylation level of the fusion in the absence of the candidate compound, identifies a compound that decreases the phosphorylation activity of level of the kinase. In a preferred embodiment, determination of the phosphorylation level is carried out using a phospho-specific antibody. In another preferred embodiment, the kinase is a tyrosine kinase or a serine/threonine kinase. More preferably, the tyrosine kinase is an abl kinase, for example, v-abl. In yet other preferred embodiments, the nucleic acid-protein fusion is either an RNA-protein fusion or a DNA-protein fusion. The compound may be any molecule, and is preferably a protein.

By “nucleic acid-protein fusion molecule” is meant a nucleic acid molecule covalently bound to a protein wherein the nucleic acid encodes the protein. The “nucleic acid” may be an RNA or DNA molecule, or may include RNA or DNA analogs at one or more positions in the sequence. The “protein” portion of the fusion is composed of two or more naturally occurring or modified amino acids joined by one or more peptide bonds. “Protein,” “peptide,” and “polypeptide” are used interchangeably herein. Typically, the protein is positioned at the 3′ or 5′ end of the nucleic acid sequence.

By “substantially pure polypeptide” or “substantially pure and isolated polypeptide” is meant a polypeptide (or a fragment thereof) that has been separated from at least some of the components that accompany it in its natural state. Typically, the polypeptide is substantially pure when it is at least 60%, by weight, free from the proteins and naturally-occurring organic molecules with which it is naturally associated. Preferably, the polypeptide is a kinase target or kinase inhibitor polypeptide that is at least 75%, more preferably, at least 90%, and most preferably, at least 99%, by weight, pure. A substantially pure kinase target or inhibitor polypeptide may be obtained, for example, by extraction from a natural source (e.g., a cell), by expression of a recombinant nucleic acid encoding a kinase target or inhibitor polypeptide, or by chemically synthesizing the polypeptide. Purity can be measured by any appropriate method, e.g., by column chromatography, polyacrylamide gel electrophoresis, or HPLC analysis.

By a “substantially pure nucleic acid,” “isolated nucleic acid,” or “substantially pure and isolated nucleic acid” is meant nucleic acid (for example, DNA) that is free of the genes that, in the naturally-occurring genome of the organism from which the nucleic acid of the invention is derived, flank the nucleic acid. The term therefore includes, for example, a recombinant DNA that is incorporated into a vector; into an autonomously replicating plasmid or virus; or into the genomic DNA of a prokaryote or eukaryote; or that exists as a separate molecule (e.g., a cDNA or a genomic or cDNA fragment produced by PCR or restriction endonuclease digestion) independent of other sequences. It also includes a recombinant DNA that is part of a hybrid gene encoding additional polypeptide sequence.

By an “abl kinase target polypeptide” is meant a polypeptide that is phosphorylated by an abl kinase. The abl kinase may be, for example, v-abl, which is available from a number of commercial sources, including New England Biolabs (Beverly, Mass.), and can also be produced using standard recombinant DNA techniques. The phosphorylation of a polypeptide by an abl kinase may be detected, for example, using antibody binding assays, including phospho-specific antibody binding assays, described herein, or any other assay known to one skilled in the art. In addition, an abl kinase target polypeptide may be contained in the protein portion of a nucleic acid protein fusion molecule.

By “abl kinase activity” is meant a biological activity mediated by an abl kinase polypeptide. The abl kinase may be, for example, v-abl. Biological activities mediated by an abl kinase polypeptide include, but are not limited to, phosphorylation of an abl kinase target polypeptide, which can be detected, for example, using the assays described herein.

By a “compound,” “test compound,” or “candidate compound” is meant a chemical molecule, be it naturally-occurring or artificially-derived, and includes, for example, peptides, proteins, synthetic organic molecules, naturally-occurring organic molecules, nucleic acid molecules, and components thereof.

By “substantially identical” is meant a nucleic acid molecule or polypeptide exhibiting at least 50%, preferably, at least 60%, more preferably, at least 70%, still more preferably, at least 80%, and most preferably, at least 90% identity to a reference nucleic acid sequence or polypeptide, respectively. For comparison of nucleic acid molecules, the length of sequences for comparison will generally be at least 30 nucleotides, preferably, at least 50 nucleotides, more preferably, at least 60 nucleotides, and most preferably, the full length nucleic acid molecule. For comparison of polypeptides, the length of sequences for comparison will generally be at least 10 amino acids, preferably, at least 15 amino acids, more preferably, at least 20 amino acids, and most preferably, the full length polypeptide.

The “percent identity” of two nucleic acid or polypeptide sequences can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, Academic Press, 1987; and Sequence Analysis Primer, Gribskov, and Devereux, eds., M. Stockton Press, New York, 1991; and Carillo and Lipman, SIAM J. Applied Math. 48:1073, 1988.

Methods to determine identity are available in publicly available computer programs. Computer program methods to determine identity between two sequences include, but are not limited to, the GCG program package (Devereux et al., Nucleic Acids Research 12:387, 1984), BLASTP, BLASTN, and FASTA (Altschul et al., J. Mol. Biol. 215:403, 1990). The well known Smith Waterman algorithm may also be used to determine identity. The BLAST program is publicly available from NCBI and other sources (BLAST Manual, Altschul, et al., NCBI NLM NIH Bethesda, Md. 20894). Searches can be performed in URLs such as the following

http://www.ncbi.nlm.nih.gov/BLAST/unfinishedgenome.html; or http://www.tigr.org/cgi-bin/BlastSearch/blast.cgi. These software programs match similar sequences be assigning degrees of homology to various substitutions, deletions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

By a “solid support” is meant any solid surface including, without limitation, any chip (for example, silica-based, glass, or gold chip), glass slide, membrane, bead, solid particle (for example, agarose, Sepharose, polystyrene or magnetic bead), column (or column material), test tube, or microtiter dish.

By a “microarray” or “array” is meant a fixed pattern of immobilized objects on a solid surface or membrane. As used herein, the array is made up of polypeptides, cDNAs, or ESTs immobilized on the solid surface or membrane. “Microarray” and “array” are used interchangeably. Preferably, the microarray has a density of between 10 and 1,000 objects/cm².

By “detectably-labeled” is meant any means for marking and identifying the presence of a molecule, wherein the molecule may be, for example, an oligonucleotide probe or primer, a gene or fragment thereof, a cDNA molecule, or an antibody. Methods for detectably-labeling a molecule are well known in the art and include, without limitation, radioactive labeling (e.g., with an isotope such as ³²P or ³⁵S) and nonradioactive labeling (e.g., with a fluorescent label, such as fluorescein, or a chemiluminescent label).

By “high stringency conditions” is meant conditions that allow hybridization comparable with the hybridization that occurs using a DNA probe of at least 500 nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. (these are typical conditions for high stringency Northern or Southern hybridizations). High stringency hybridization is also relied upon for the success of numerous techniques routinely performed by molecular biologists, such as high stringency PCR, DNA sequencing, single strand conformational polymorphism analysis, and in situ hybridization. In contrast to Northern and Southern hybridizations, these techniques are usually performed with relatively short probes (e.g., usually 16 nucleotides or longer for PCR or sequencing, and 40 nucleotides or longer for in situ hybridization). The high stringency conditions used in these techniques are well known to those skilled in the art of molecular biology, and may be found, for example, in Ausubel et al., Current Protocols in Molecular Biology, John Wiley & Sons, New York, N.Y., 1998, hereby incorporated by reference.

By “transgene” is meant any piece of DNA that is inserted by artifice into a cell and becomes part of the genome of the organism that develops from that cell. Such a transgene may include a gene that is partly or entirely heterologous (i.e., foreign) to the transgenic organism, or may represent a gene homologous to an endogenous gene of the organism. By “transgenic cell” is meant any cell that includes a DNA sequence that is inserted by artifice into a cell and becomes part of the genome of the organism that develops from that cell. As used herein, the transgenic organisms are generally transgenic mammals (e.g., mice, rats, and goats) and the DNA (transgene) is inserted by artifice into the nuclear genome.

By “knockout mutation” is meant an artificially-induced alteration in the nucleic acid sequence (created via recombinant DNA technology or deliberate exposure to a mutagen) that reduces the biological activity of the polypeptide normally encoded therefrom by at least 80% relative to the unmutated gene. The mutation may, without limitation, be an insertion, deletion, frameshift mutation, or a missense mutation. The knockout mutation can be in a cell ex vivo (e.g., a tissue culture cell or a primary cell) or in vivo. A “knockout animal” is a mammal, preferably, a mouse, whose cells contain a knockout mutation as defined above.

By “transformation,” “transfection,” or “transduction” is meant any method for introducing foreign molecules into a cell, e.g., a bacterial, yeast, fungal, algal, plant, insect, or animal cell. Lipofection, DEAE-dextran-mediated transfection, microinjection, protoplast fusion, calcium phosphate precipitation, retroviral delivery, electroporation, and biolistic transformation are just a few of the methods known to those skilled in the art which may be used. In addition, a foreign molecule can be introduced into a cell using a cell penetrating peptide, for example, as described by Fawell et al. (Proc. Natl. Acad. Sci. U.S.A. 91:664-668, 1994) and Lindgren et al. (TIPS 21:99-103, 2000). By “transformed cell,” “transfected cell,” or “transduced cell,” is meant a cell (or a descendent of a cell) into which a nucleic acid molecule encoding a polypeptide of the invention has been introduced, by means of recombinant nucleic acid techniques.

By “promoter” is meant a minimal sequence sufficient to direct transcription. If desired, constructs of the invention may also include those promoter elements that are sufficient to render promoter-dependent gene expression controllable in a cell type-specific, tissue-specific, or temporal-specific manner, or inducible by external signals or agents; such elements may be located in the 5′ or 3′ or intron sequence regions of the native gene.

By “operably linked” is meant that a gene and one or more regulatory sequences are connected in such a way as to permit gene expression when the appropriate molecules (e.g., transcriptional activator proteins) are bound to the regulatory sequences.

By “modulating” is meant either increasing (“upward modulating”) or decreasing (“downward modulating”) the activity of a kinase in vivo or ex vivo. It will be appreciated that the degree of kinase activity provided by a modulatory compound in a given assay will vary, but that one skilled in the art can determine the statistically significant change in the level of kinase activity that identifies a compound that increases or decreases kinase activity. Kinase activity can be measured, for example, as described above.

Preferably, for kinase inhibitors, kinase activity is decreased by at least 20%, more preferably, by at least, 40%, 50%, or 75%, and, most preferably, by at least 90%, relative to a control sample which was not administered a kinase inhibitor. Also as used herein, preferably, for upward modulating, kinase activity is increased by at least 1.5-fold to 2-fold, more preferably, by at least 3-fold, and most preferably, by at least 5-fold, relative to a control sample which was not administered a kinase upward modulating test compound.

By a “purified antibody” is meant an antibody that is at least 60%, by weight, free from proteins and naturally occurring organic molecules with which it is naturally associated. Preferably, the preparation is at least 75%, more preferably, at least 90%, and, most preferably, at least 99%, by weight, antibody, e.g., an abl kinase target polypeptide-specific antibody. A purified antibody may be obtained, for example, by affinity chromatography using recombinantly-produced protein or conserved motif peptides and standard techniques.

By “specifically binds” is meant a compound that recognizes and binds a protein or polypeptide, for example, a kinase target or inhibitor polypeptide, and that, when detectably labeled, can be competed away for binding to that protein or polypeptide by an excess of compound that is not detectably labeled. A compound that non-specifically binds is not competed away by the above excess unlabeled compound. Desirably, an antibody that “specifically binds” has an affinity, for example, for an abl kinase target polypeptide that is at least 2-fold, at least 5-fold, at least 10-fold, at least 30-fold, or at least 100-fold greater than for an equal amount of any other protein.

The general approach of the present invention provides a number of advantages for identifying cellular targets of kinases and their inhibitors. For example, direct mRNA display allows the rapid and efficient mapping of protein-protein interactions, through the generation of RNA-protein fusion molecules, an approach which is useful for drug screening. Because the link between the protein and its genotype (RNA) is covalent, the protein moiety can be selected under robust conditions and its genetic material can be amplified by PCR in an entirely in vitro system (Roberts and Szostak, Proc. Natl. Acad. Sci. U.S.A. 94:12297-302, 1997; and Roberts, Curr. Opin. Chem. Biol. 3:268-73, 1999).

In addition, as mRNA display is a completely in vitro technique, many of the problems inherent in cloning and expression are eliminated. Because library synthesis and selection occur entirely in vitro, it is possible to minimize the loss of genetic information normally associated with the subcloning and transformation steps of other protein display systems such as yeast two hybrid and phage display. Moreover, the formation of mRNA display constructs is readily achieved in a mammalian expression system, thereby providing suitable chaperones for the folding of human proteins and the potential for appropriate post-translational modifications.

The in vitro nature of the system also makes it ideally suited for the identification of target sequences undergoing specific post-translational modifications. When used in conjunction with protein structural information and gene expression profiling, this technology provides a critical element for the post-genomic analysis of the proteome.

In addition, cellular mRNA-protein libraries offer several advantages for the identification of novel kinase-substrate interactions. First, since they are derived from cellular mRNA they reflect the cell's proteome and therefore consist of a pool of biologically relevant targets. Second, since the libraries are of cellular origin, clones enriched during the selection can be efficiently analyzed by hybridization to cDNA microarray filters analogous to the methods used to quantify cellular transcript levels. Third, the relatively limited number of mRNAs in a cell, coupled with the large size of the library, allows for multiple representation of each protein in the starting library. And finally, because a random priming approach is used during cDNA synthesis of the mRNA-protein fusion library, minimal interacting domains of interest can be mapped from overlapping clones enriched during the selection.

As applied to the identification of abl kinase targets, the invention also has several important utilities. For example, abl kinases have been implicated in a number of cancers. Therefore, abl kinase target polypeptides can be used in screens for therapeutics that modulate diseases or developmental abnormalities involving overactivity or underactivity of abl kinases, such as cell death, proliferation, and differentiation pathways. Polypeptides that are targets of abl kinases, as described herein, may be used as targets in therapeutic screening assays. The polypeptides identified as targets of abl kinases may also be used to detect abl kinases in a sample. In addition, the methods of the present invention are useful as high-throughput screening methods for potential therapeutics that can be used to counteract overactivity or underactivity of abl kinases, for example, for the treatment of diseases or disorders involving cell death, proliferation, or differentiation.

### DETAILED DESCRIPTION

The drawings will first briefly be described.

### BRIEF DESCRIPTION OF THE DRAWINGS

FIG. 1 is a schematic drawing of the procedure used to select targets of v-abl from RNA-protein fusion molecule libraries. In the first round of the selection, purified mRNA-protein fusion molecules were phosphorylated by v-abl and subjected to immunoprecipitation with the 4G10 antibody. The mRNA-protein fusion molecules were eluted from the antibody using KOH and the cDNAs amplified by PCR. In subsequent rounds, the mRNA-protein pool was pre-cleared with the α4G10/protein A Sepharose beads prior to the kinase reaction to remove any nonspecific binders, as well as mRNA-protein fusions undergoing non-specific tyrosine phosphorylation during in vitro translation.

FIG. 2A is a graph of the percent elution of the randomized RNA-protein fusion library following six rounds of v-abl phosphorylation and 4G10 antibody immunoprecipitation. The percent elution was based on the input of RNA-protein fusion molecules labeled with [³⁵S]-methionine. In round one, the library was not pre-cleared prior to phosphorylation with v-abl.

FIG. 2B is a phosphorimage of the phosphorylation of v-abl substrates by v-abl. DNA from the starting library (lane 3) and the post-round six elution (lane 4) was transcribed by T7 polymerase, translated in the absence of radiolabeled methionine and immunoprecipitated with anti-FLAG protein A Sepharose beads. In the kinase reactions, immunoprecipitates were phosphorylated with v-abl in the presence of [γ-³²P]ATP, washed extensively, and then resolved on a 4-20% Tris-glycine gel. As a control for immunoprecipitation, parallel in vitro translation reactions were done in the presence of [³⁵S]-methionine. The B43 clone is a negative control for the kinase reaction (lane 2), and contains a single tyrosine residue in the FLAG epitope (DYKDDDDK), while the C23 clone serves as the positive control (lane 1) and contains the known v-abl phosphorylation sequence (IYAAP).

FIG. 3A is a table showing phosphorylation consensus sequences identified as v-abl target polypeptides after six rounds of selection, with respect to the fixed tyrosine residue (SEQ ID NOS: 1-20). Amino acids immediately surrounding the randomized sequences are shown. The number of sequences in each category is indicated within the brackets. Sequences tested for phosphorylation by v-abl are shown.

FIG. 3B is a table showing phosphorylation consensus sequences identified as v-abl target polypeptides after six rounds of selection, with respect to additional tyrosine residues introduced within the randomized region by the selection (SEQ ID NOS: 2, 20, 21, 16, 10, 5, 18, 11, 12, 15). The number of clones in each category is indicated directly adjacent to the consensus sequence. The number of sequences within each category where the novel tyrosine is part of the I/L/V-Y-X₁₋₅-P/F (SEQ ID NO: 108) consensus sequence is indicated at the far right. Sequences tested for phosphorylation by v-abl are shown.

FIG. 3C is a table showing sequences identified after six rounds of phosphorylation by v-abl kinase and immunoprecipitation by 4G10 antibody in a selection assay. These sequences lack a tyrosine or an obvious v-abl phosphorylation motif (SEQ ID NOS: 22-32).

FIG. 4A is a set of scanned images of cDNA microarray filters showing the hybridization of radiolabeled DNA from round zero (left panel) or round four (right panel). Hybridization of the pools to one of the filter's six grids is shown. Examples of increased signals following hybridization of the round four pool relative to the round zero are indicated.

FIG. 4B is a table of the clones identified by the DNA microarray analysis as targets of v-abl kinase, as well as their GenBank Accession numbers and potential v-abl phosphorylation sites (SEQ ID NOS: 33-44).

FIG. 4C is a table of genes identified by sequence analysis following round four (Rd4) or five (Rd5) of the v-abl selection, as well as the name of the parent gene identified by a BLAST homology search, and the insert sequence that was identified in the screen for targets of v-abl (SEQ ID NOS: 45-58). Relevant tyrosine residues and surrounding amino acids are underlined and in bold type for each identified sequence.

FIG. 5A is a sequence comparison between TC26 and other members of the SH2-domain adaptor protein family (SEQ ID NOS: 59-64). Potential v-abl phosphorylation sites are indicated.

FIG. 5B shows the peptide sequences used to map the tyrosine residue phosphorylated by v-abl (SEQ ID NOS: 65-72).

FIG. 5C is a scanned image of v-abl kinase phosphorylation of peptides encoded by the TC26 clone. Phosphorylation reactions were done in the presence of recombinant v-abl and [γ-³²P]ATP either in the absence (−) of substrate or, using the optimal v-abl target peptide (+), or one of the peptides shown in FIG. 5B.

FIGS. 5D and 5E show the full length Shg amino acid (SEQ ID NO: 73) and nucleic acid (SEQ ID NO:74) sequences. The original sequence obtained following five rounds of selection (i.e., the minimal sequence shown to be phosphorylated by v-abl in vitro) is boxed (SEQ ID NOS: 108, 109), with the tyrosine substrate of v-abl underlined and in bold. A putative myristylation site and an SH2 domain are indicated in FIG. 5D by a dashed and solid underline respectively; the asterisk denotes the stop codon.

FIG. 6A is a table of the sequences of the phenylalanine derivatives tested for inhibition of v-abl kinase activity (SEQ ID NOS: 75-94), as well as their isoelectric points. Mutagenized test peptides (m-series) as well as the scrambled controls (r-series) are shown. Relative v-abl activity was scored as: 90-100% (−), 50-90% (+), or 10-50% (++). A subset of control peptides (r-series) containing the same amino acids as the test peptides, but in a scrambled order, did not have a significant effect on v-abl kinase activity.

FIG. 6B is a set of scanned images of examples of kinase inhibition assays. Mutagenized peptides m-M16, m-B47, or r-B47 were added to kinase reactions containing an optimized v-abl substrate at a 100:1, 10:1, or 1:1 molar ratio relative to the target peptide. Kinase reactions in the absence of an inhibitory peptide (−) were done for comparison.

FIGS. 7A-7D list targets of the v-abl kinase, and their nucleic acid sequences (SEQ ID NOS: 46-51, 55-57, 110-140).

FIG. 8 is a graph showing phosphorylation of an optimized substrate peptide by cAMP-dependent protein kinase (PKA).

FIGS. 9A and 9B illustrate experiments that demonstrate that ligand-activated PDGF receptor binds the SH2 domain of the Shg polypeptide.

Described herein are methods for identifying cellular protein targets that are phosphorylated by kinases, and methods for identifying compounds that modulate the phosphorylation activity of such kinases. These methods generally make use of mRNA display (Roberts and Szostak, supra), a technique in which a DNA template is used to transcribe an engineered-mRNA molecule possessing suitable flanking sequences (e.g., a promoter, a functional 5′ UTR to allow ribosome binding, a start codon, an open reading frame, a sequence for polypeptide purification, and a conserved sequence used for ligation to a complementary linker containing a peptide acceptor, such as puromycin). To the 3′ end of the mRNA, a linker strand with a peptide acceptor is then added, preferably by photo-crosslinking. When this RNA is translated in vitro, the peptide acceptor becomes incorporated at the C-terminus of the nascent peptide.

The resulting mRNA display construct is then purified after ribosome dissociation. A cDNA strand is synthesized to protect the RNA and to provide a template for future PCR amplification. A library of such constructs can be incubated with a desired kinase, such as an abl kinase, and molecules that are phosphorylated by the kinase are enriched by immunoprecipitation with an anti-phosphotyrosine antibody and a solid support. cDNAs encoding peptides phosphorylated by the kinase are recovered from the solid support, for example, by KOH elution, and subsequent PCR is performed to regenerate a library enriched for kinase target polypeptides.

The mRNA display technique is described generally, for example, in Roberts and Szostak (Proc. Natl. Acad. Sci. U.S.A. 94:12297-302, 1997) and Szostak et al. (WO 98/31700; and U.S. Pat. No. 6,261,804 B1), hereby incorporated by reference. RNA-protein fusions can also be generated by the methods of Gold, U.S. Pat. No. 5,843,701 and U.S. Pat. No. 6,194,550. Alternatively, the methods of the present invention may be carried out using nucleic acid-protein fusion molecules that are DNA-protein fusion molecules, for example, cDNA-protein fusions. Such molecules are described, for example, in U.S. Pat. No. 6,416,950 B1 and WO 00/32823, and Kurz et al. (Chem. Biochem. 2:666-672, 2001) hereby incorporated by reference.

Techniques for carrying out each method of the invention are now described in detail, using particular examples. Also described herein are novel polypeptides identified as targets and inhibitors of abl kinase, and nucleic acid molecules that encode those targets and inhibitors. While the invention is described with respect to abl as the kinase, any kinase (including any tyrosine or serine/threonine kinase) can be used in these methods, and targets and inhibitors of that kinase identified as described below. Examples of other preferred kinases whose targets may be identified by the methods of the invention include, without limitation, the ZAP-70, Tie-2, Lyn, Fyn, and Src kinases.

The following examples are provided for the purpose of illustrating the invention, and should not be construed as limiting.

### EXAMPLE 1

Methods for Identifying Cellular Kinase Targets and Selection of Cellular Proteins Phosphorylated by the Abl Kinase

Model system

Substrate sequences surrounding a phosphorylated residue are important in determining protein kinase specificity. Consequently, we sought to identify kinase substrates from a randomized peptide library and a cellular proteomic library displayed as mRNA-protein fusions. In a first experiment, a control mRNA-protein fusion molecule containing an optimized v-abl phosphorylation site (EAIYAAPFAKKK; SEQ ID NO: 95, New England Biolabs, Beverly, Mass.) was synthesized. Fusions were purified from the reverse transcription reaction by oligo d(T) chromatography, phosphorylated with recombinant v-abl, and immunoprecipitated with 4G10 antibodies, which are phosphotyrosine-specific (Upstate Biotechnology, Lake Placid, N.Y.), according to the manufacturer's directions. Approximately 3% of the control mRNA-peptide fusion molecules were immunoprecipitated by the 4G10 antibody following phosphorylation by v-abl. Immunoprecipitation was specific for the phosphotyrosine-specific antibody and absolutely dependent upon recombinant v-abl.

Together these results demonstrated that v-abl was functional at substrate concentrations (50-100 nM) significantly below the Kₘ of the enzyme (50 μM), and that the translational lysates did not contain significant endogenous v-abl activity. We reasoned therefore that repeated rounds of phosphorylation of a mRNA-protein fusion library by v-abl, followed by selection of tyrosine-phosphorylated fusions with the 4G10 antibody, could be used to identify substrate targets of the v-abl oncogene.

Enrichment of mRNA-peptide fusions containing phosphorylated tyrosines

To identify peptide substrates of the v-abl tyrosine kinase, we synthesized an mRNA-peptide fusion library in which an invariant tyrosine was flanked by five randomized amino acids (GCGGX₅-Y-X₅GCG; SEQ ID NO: 96). The randomized peptide library was constructed as follows. Approximately 1.2 nmoles of the gel-purified Abl-2NNS oligo (GGC GAG GAG GGA TGT GGA GGA NNS₅ TAC NNS₅ GGA TGT GGA GAC TAC AAG GAC GAG) (SEQ ID NO: 97), encoding the amino acid sequence GEEGCGGX₅YX₅GCGDYKDE (SEQ ID NO: 98), was amplified by PCR (3.5 ml) using the f-Abl-NNS (forward) primer (TAA TAC GAC TCA CTA TAG GGA CAA TTA CTA TTT ACA ATT ACA ATG GTG AGC AAG GGC GAG GAG GGA TGT GGA GG) (SEQ ID NO: 99) and the r-Abl-NNS (reverse) primer (AGC TTT TGG CTC GTC CTT GTA GTC TCC) (SEQ ID NO: 100). The forward primer was designed to incorporate sequences required for in vitro transcription and translation while the reverse primer was used to add sequences needed for RNA-DNA ligation and fusion production.

The library was amplified through 5 cycles of PCR in a microtiter plate containing Ready-To-Go™ PCR beads (Amersham Pharmacia, Piscataway, N.J.) under the following conditions: 95° C. for 1 minute, 65° C. for 2 minutes, and 72° C. for 1 minute, followed by a 5 minutes extension step at 72° C. PCR products were extracted with phenol/chloroform/iso-amyl alcohol, concentrated by ethanol precipitation, and transcribed using the T7 polymerase MEGAshortscript™ in vitro transcription kit, according to the manufacturer's direction (Ambion, Austin, Tex.).

Following treatment with DNase, the RNA was phenol extracted, purified on a NAP 25 column (Amersham Pharmacia), and concentrated by isopropanol precipitation. Purified RNA was then ligated to a DNA-puromycin linker (p-dA₂₈CCPu) using the SPL-PKA oligo (TTT TTT TTT TNA GCT TTT GGC TCG TC) (SEQ ID NO: 101) as a splint between the 3′ terminus of the RNA and the 5′ terminus of the puromycin linker. The RNA, DNA-puromycin linker, and splint (35 nmoles each) were heated to 70° C. for 3 minutes and incubated at room temperature prior to the addition of 10× ligation buffer and T4 ligase (Promega, Madison, Wis.). The ligation mixture was resolved on a polyacrylamide-urea denaturing gel (NOVEX, San Diego, Calif.) and the ligated product was eluted using a buffer containing 200 mM NaCl, 10 mM Tris, pH 7.4, and 1 mM EDTA.

Following ethanol precipitation, 1 nmole of ligated RNA was added to a 1.8 ml Retic lysate IVT™ translation reaction (Ambion) containing rabbit reticulocyte lysate and [³⁵S]-methionine. Following incubation at 30° C. for 30 minutes, mRNA-protein fusion was promoted by the addition of KCl and MgCl₂ (final concentrations 500 mM and 150 mM, respectively) and incubation at 25° C. for 1 hour. mRNA-protein fusion molecules were purified from the in vitro translation mix by oligo d(T) chromatography using the poly A sequence in the DNA-puromycin linker. Briefly, the translation-fusion mix was diluted 10-fold in cold (4° C.) oligo d(T) binding buffer (100 mM Tris-HCl, pH 8.0, 1 M NaCl, 0.25% Triton X-100, 10 mM EDTA) and bound to equilibrated oligo d(T) resin (Amersham Pharmacia) in a batch format. The oligo d(T) slurry was transferred to a column and the bed was washed extensively with binding buffer lacking EDTA (4° C.). mRNA-protein fusions were eluted in water (25° C.) and quantified by scintillation counting. cDNA synthesis from the fusion library (50 pmoles) was done using the r-Abl-NNS primer and SUPERSCRIPT™ II RNase H⁻ Reverse Transcriptase (GibcoBRL Life Technologies, Grand Neck, N.Y.) as suggested by the manufacturer. Approximately 25 pmoles of the starting library was used in the first round of the selection.

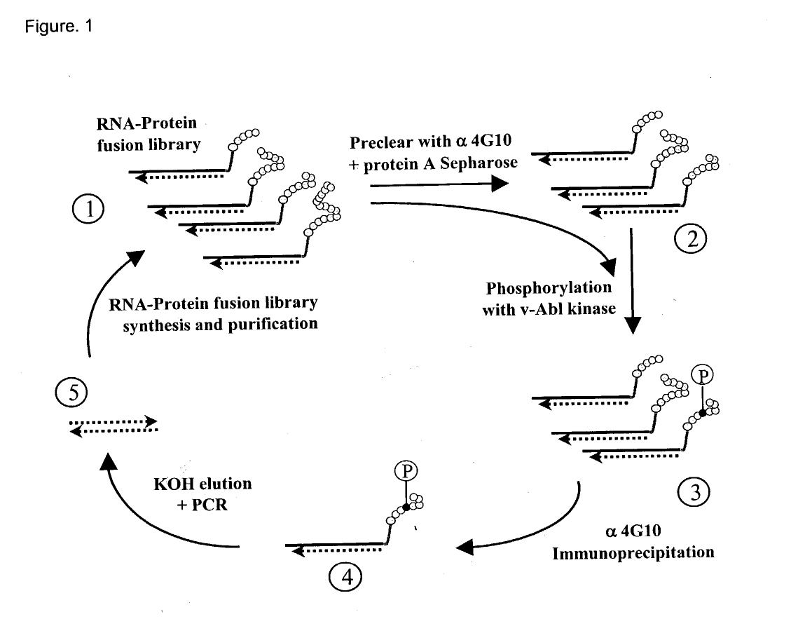

The mRNA-peptide fusions were phosphorylated by v-abl in vitro, immunoprecipitated using the phosphotyrosine-specific 4G10 antibody and the attached genetic information was amplified by PCR, as shown in FIG. 1. The phosphorylation and selection steps were carried out as follows. Following cDNA synthesis, as described above, mRNA-protein libraries were diluted 10-fold into 4G10 binding buffer (50 mM Tris-HCl, pH 7.6, 150 mM NaCl, 1% NP 40, 1 mM EGTA, 10% glycerol, 1 mM Na₃VO₄, and 1 mM NaF) and incubated with 20 μg of the 4G10 anti-phosphotyrosine antibody at 4° C. for 2 hours. Pre-equilibrated protein A Sepharose beads (400 μl, 50% slurry, Amersham Pharmacia) were added to the immunoprecipitation buffer and incubation continued for 1 hour. The non-bound void fraction was diluted 2-fold in oligo d(T) binding buffer and purified on an oligo d(T) column as described previously. Peak fractions (10-20 pmoles) were added directly to kinase reactions (3 hours at 30° C.) containing recombinant v-abl (New England Biolabs, Beverly, Mass.) and 100 μM ATP, under conditions described by the manufacturer.

Phosphorylated targets were immunoprecipitated using the 4G10 antibody and protein A Sepharose beads, and the beads were washed five times with the immunoprecipitation buffer. Bound complexes and encoding cDNAs were eluted with 0.1 N KOH (4×100 μl) and neutralized with 1/10 volume of a 1 M Tris-HCl, pH 8.0 and acetic acid solution prior to PCR. RNA-protein fusion molecules were quantified by scintillation counting in the presence of scintillation fluid. In round one of the selection, the pre-clear step was omitted and the mRNA-protein fusion libraries were added to the kinase reactions after the reverse transcription step and purification on an oligo d(T) column. Immunoprecipitation and recovery of cDNA from the bound complexes was achieved as described above.

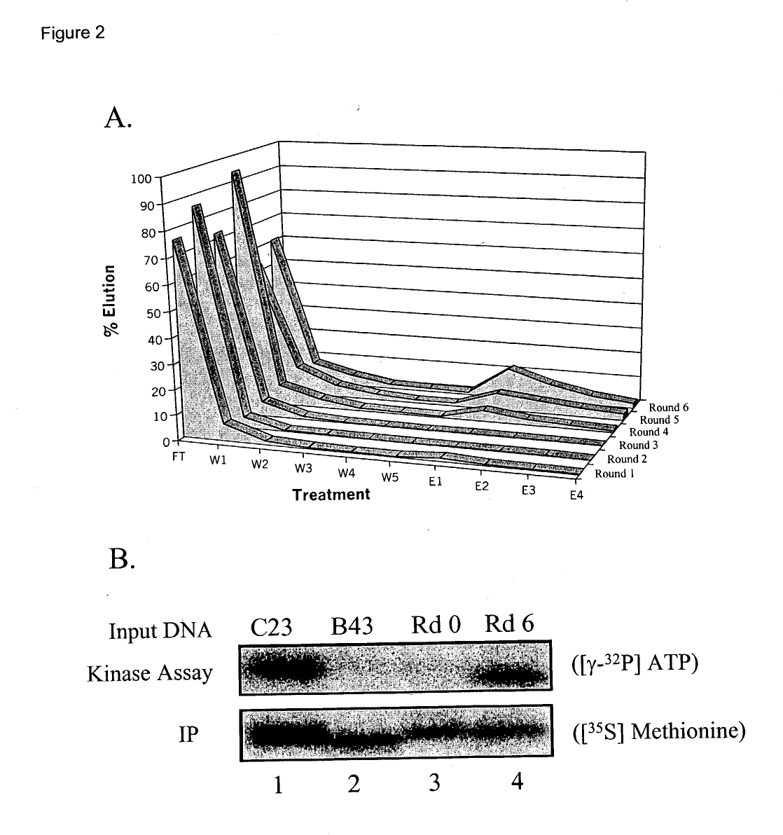

In these studies, we anticipated that the starting mRNA-peptide library (25 pmoles) should contain approximately one copy of each possible sequence (20¹⁰ members). In order to minimize the loss of genetic diversity, the starting library was not pre-cleared in the first round of the selection and approximately 1.5% of the input mRNA-peptide fusion molecules were eluted (fractions E1+E2) from the 4G10 antibody in round one (FIG. 2A). In subsequent rounds, the library was pre-cleared by incubating it with 4G10 antibody/protein A Sepharose beads prior to phosphorylation with v-abl, as described above, in order to remove non-specific binders and any protein domains having tyrosines that were fortuitously phosphorylated during the in vitro translation reaction. In rounds two and three, the elutions did not yield detectable radioactive counts, although subsequent PCR reactions yielded genetic material that was carried on to the next round. In contrast, approximately 6.5% of the input mRNA-peptide fusion molecules were eluted (fractions E1+E2) from the 4G10 antibody in round four, and this increased to almost 20% in round six. PCR analysis demonstrated that the last wash of each round was essentially devoid of genetic material.

In order to confirm the enrichment of v-abl substrates, the DNAs from the starting library and the post-round six pool were expressed as free peptides and tested for phosphorylation by v-abl (FIG. 2B). Briefly, unligated RNA (approximately 50 pmoles) was in vitro translated in the absence of [³⁵S]-methionine. FLAG-tagged peptides were immunoprecipitated with M2-FLAG agarose beads (Sigma, St. Louis, Mo.), and equilibrated in v-abl kinase buffer. Phosphorylation reactions were done in the presence of [γ-³²P]ATP essentially as suggested by the manufacturer. Free [γ-³²P]ATP was removed by washing the beads in kinase buffer. Phosphorylated products were resolved on a 4-12% BIS-Tris gel with MES as the running buffer and visualized by the STORM 860 phosphorimaging system (Molecular Dynamics, Sunnyvale, Calif.). To confirm the immunoprecipitation of the desired peptides, parallel immunoprecipitations were done using peptides labeled with [³⁵S]-methionine.

In this assay, no phosphorylation of the starting peptide pool was detected, presumably due to the low abundance of v-abl target sequences (lane 3). In contrast, phosphorylation of the post-round six peptide pool was readily observed (lane 4), demonstrating the enrichment of target sequences. A peptide containing a single tyrosine residue in the FLAG epitope (DYKDDDDK; SEQ ID NO: 8) was not phosphorylated by v-abl (lane 2) demonstrating that phosphorylation of the test peptides was sequence-specific, while phosphorylation of a control fusion molecule containing a known v-abl recognition motif was readily detected (lane 1). Together these results demonstrate that substrates of v-abl were enriched from the mRNA-peptide library following six rounds of selection and PCR amplification of genetic material.

Sequence analysis of the abl kinase targets

Sixty-nine clones from the post-round six pool described above were sequenced according to the following methods. The round six PCR products were cloned into the pCR 2.1-TOPO vector and transformed into TOP10 cells, as recommended by the manufacturer (Invitrogen, Carlsbad, Calif.). DNA was prepared using the Qiaprep Spin miniprep kit (Qiagen), and inserts were sequenced using standard methods. The BLAST and ClustalW⁶⁰ programs were used to search the GenBank database and align protein sequences, respectively.

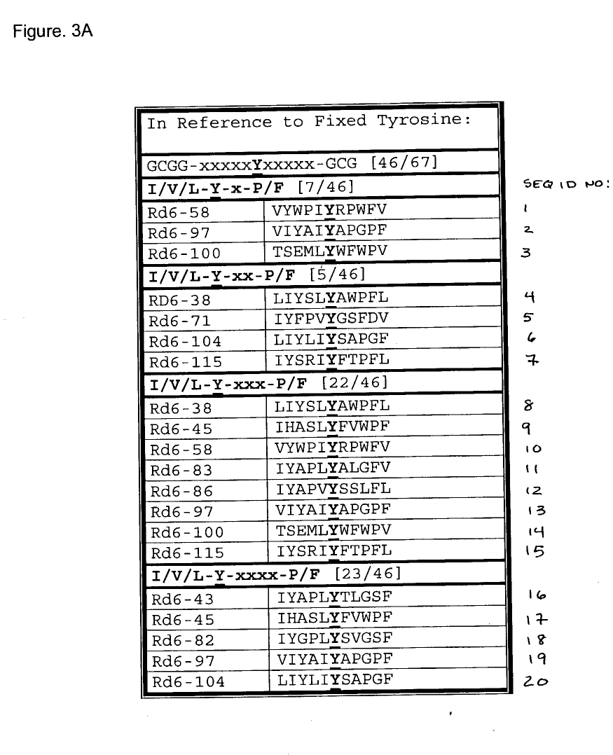

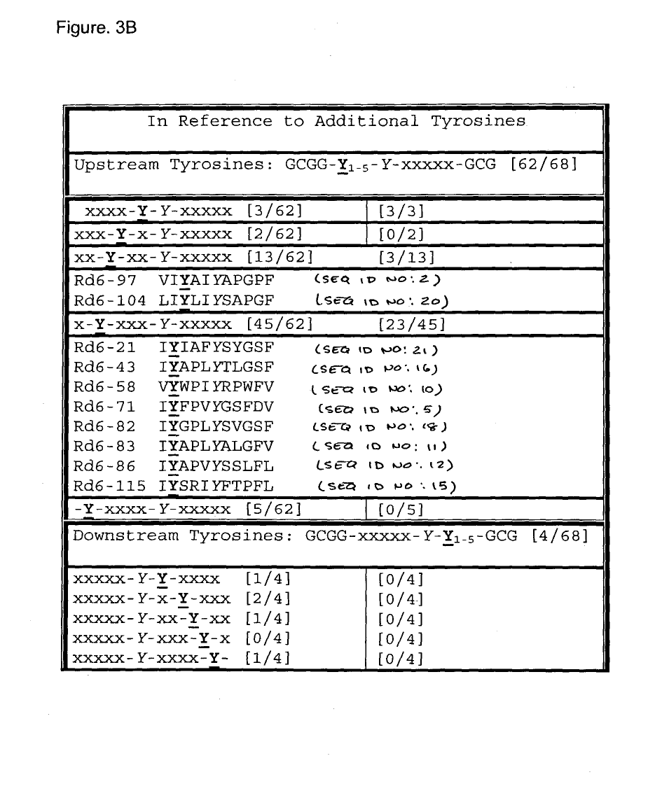

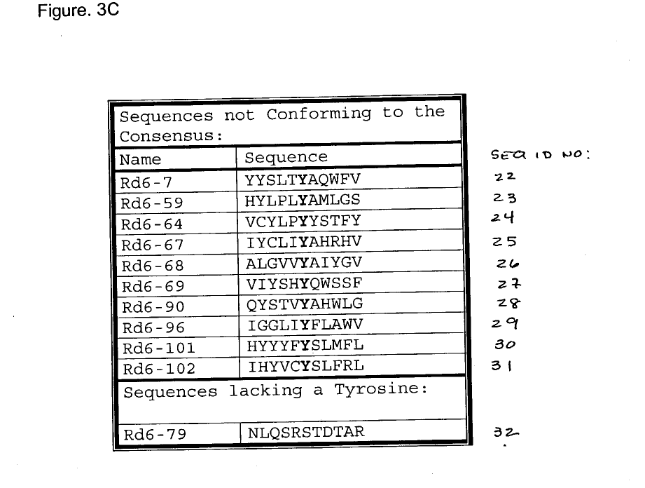

Sequence analysis of the 69 clones showed that the invariant tyrosine was missing in two of the 69 clones. In one of these cases, a novel tyrosine was introduced upstream of the invariant residue while in the other case, there were no tyrosine residues. Of the remaining 67 clones, 46 matched the I/L/V-Y-X₁₋₅-P/F consensus motif with respect to the invariant tyrosine (FIG. 3A). The majority of these clones had a proline or phenylalanine residue four (22/46) or five (23/46) amino acids downstream of the invariant tyrosine.

In most clones (66/68), a second tyrosine was introduced into the random region either upstream (62) or downstream (4) of the fixed residue (FIG. 3B). Of the 21 clones in which the invariant tyrosine was not part of a consensus sequence, 11 contained a second tyrosine that did match the I/L/V-Y-X₁₋₅-P/F consensus. In the majority of clones, an upstream tyrosine was introduced either three (13/62) or four (45/62) residues upstream of the fixed tyrosine. Only 3/13 clones containing the tyrosine (y) at position N=−3 relative to the fixed residue (Y) (y-X-I/L/V-Y) conformed to the consensus phosphorylation sequence. This result is not surprising given the requirement for a hydrophobic residue upstream of the invariant tyrosine. In contrast, if the tyrosine (y) was present at N=−4, almost half the clones matched the consensus sequence (23/45). In addition, all of the clones containing an additional tyrosine immediately upstream of the fixed residue (N=1) or at positions N=−2 or N=−5, matched the v-abl kinase consensus motif with respect to either the novel or invariant tyrosine. Only 11 clones were either missing a tyrosine or contained one that did not match an obvious phosphorylation consensus motif (FIG. 3C).

In summary, 58/69 clones contained a tyrosine within the I/L/V-Y-X₁₋₅-P/F consensus motif. The v-abl kinase phosphorylated all of the clones (12/12) representing the various classes of consensus motifs that were tested (FIGS. 3A and 3B). These results demonstrated the enrichment of v-abl substrates from the randomized peptide library and suggested that v-abl can potentially phosphorylate a wider range of peptide substrates than previously thought. In addition, many of the peptides selected in this assay contained more than one tyrosine. In some cases the novel tyrosine was not part of the consensus sequence and therefore its presence may not be significant. However in other cases, dual phosphorylation of the tyrosine residues may have aided in selection of the target. The presence of cysteines flanking the randomized region allows for the possibility that the randomized peptides can be exhibited as part of a constrained surface loop during the selection. Although impossible to rule out, this seems rather unlikely given the presence of DTT in the v-abl kinase reaction and absence of cysteine residues in the selected clones.

Selection of cellular proteins phosphorylated by v-abl

As shown above, peptide substrates of v-abl were successfully selected from a randomized mRNA-peptide fusion library following six rounds of selection. In order to identify cellular targets of v-abl, we screened a proteomic mRNA-protein library derived from human bone marrow cells. A cellular mRNA-protein fusion library was constructed by random priming cellular mRNA to create a mixture of full-length and partial cDNA fragments. Specifically, poly-A⁺ mRNA from human bone marrow (Clontech, Palo Alto, Calif.) was primed using the R-HBM1 oligonucleotide GCC TTA TCG TCA TCG TCC TTG TAG TCG AAA CTA GAN₉ (SEQ ID NO: 102) and cDNA synthesized using SuperScript II RT (Promega, Madison, Wis.). After RNase H treatment, unextended primer was removed by purification over an S-300 (Amersham Pharmacia) size exclusion column equilibrated in TE (10 mM Tris-HCl, pH 8.0, 1 mM EDTA). Second strand cDNA synthesis by the Klenow fragment of E. coli DNA polymerase was primed using the F-HBM2 oligonucleotide GGA CAA TTA CTA TTT ACA ATT ACA ATG N₉ (SEQ ID NO: 103). Unextended primers were again removed using an S-300 column and the cDNA products amplified by PCR using the primer pairs TAA TAC GAC TCA CTA TAG GGA CAA TTA CTA TTT ACA ATT (T7TMVUTR) and AGA AGA TGC GCG ATC GTC ATC GTC CTT GTA GTC (FLAGRASS) (SEQ ID NOS: 104, 105). Taq polymerase (Promega, Madison, Wis.) was added to PCR reactions (1.2 ml) after an initial 5 minutes denaturation step at 95° C. An annealing temperature of 44° C. (2 minutes) was used in the first four rounds of the PCR reaction, followed by 65° C. in the subsequent rounds (20-30 cycles). Denaturation and extension steps were done at 95° C. (1 minute) and 72° C., (2 minutes) respectively, followed by a final 10 minute extension step at 72° C.

PCR products were concentrated using the QIAquick PCR purification kit (Qiagen) and fractionated on an S-300 Sephadex column. DNA from the first two fractions (500 μl) was ethanol precipitated. RNA (approximately 5 nmoles) was synthesized using the T7 polymerase MEGAscript in vitro transcription kit (Ambion) according to the manufacturer's suggestions. RNA (approximately 3 nmoles) was ligated to 4.5 nmoles of the p-dA₂₈CCPu puromycin linker using the biotinylated SPLINTRASS oligo (4.5 nmoles, B2B1GCA ACG ACC AAC TTT TTT TTT N) (SEQ ID NO: 106) as the splint. The RNA, DNA-puromycin linker, and splint were heated to 80° C. for 10 minutes and then cooled to 20° C. (0.1° C./minute) prior to the addition of 10× ligation buffer and T4 ligase (Promega). Reactions were incubated at 20° C. overnight, and then diluted to 1 ml in PBS buffer.

Neutra-Avidin beads (600 μl; Pierce, Rockford, Ill.) were then added to the mixture and incubated for 1 hour at 30° C. The beads were washed three times in pre-warmed PBS (30° C.). Ligated RNA was dissociated from the biotinylated splint by resuspending the Neutra-Avidin beads in an equal volume of water and incubating at 45° C. for 15 minutes (repeated twice). Approximately 200 pmoles of ligated RNA was recovered in a typical reaction.

Following ethanol precipitation of the RNA, fusion production and cDNA synthesis were done as described above for the random peptide library, except that the RT-RASS oligo (TTT TTT AGA AGA TGC GCG ATC GTC A) (SEQ ID NO: 107) was used as the primer. The selection was initiated with approximately 1 pmole of the library. The fusion molecules in this library were expected to be represented in sizes ranging from epitope size to full-length.

Cellular targets of v-abl were selected from this library following phosphorylation by v-abl and immunoprecipitation with the 4G10 anti-phosphotyrosine antibody. Phosphorylation by v-abl was carried out as follows. mRNA-protein libraries were diluted 10-fold into 4G10 binding buffer (50 mM Tris-HCl, pH 7.6, 150 mM NaCl, 1% NP 40, 1 mM EGTA, 10% glycerol, 1 mM Na₃VO₄, and 1 mM NaF) and incubated with 20 μg of the 4G10 antibody at 4° C. for 2 hours. Pre-equilibrated protein A Sepharose beads (400 μl, 50% slurry, Amersham Pharmacia) were added to the immunoprecipitation buffer and incubation was continued for 1 hour. The non-bound void fraction was diluted 2-fold in oligo d(T) binding buffer and purified on an oligo d(T) column, as described previously. Peak fractions (10-20 pmoles) were added directly to kinase reactions (3 hours at 30° C.) containing recombinant v-abl (New England Biolabs, Beverly, Mass.) and 100 μM ATP, under conditions described by the manufacturer. Phosphorylated targets were immunoprecipitated using the 4G10 antibody and protein A Sepharose beads, and the beads were washed five times with the immunoprecipitation buffer. Bound complexes and encoding cDNAs were eluted with 0.1 N KOH (4×100 μl) and neutralized with 1/10 volume of a 1 M Tris-HCl, pH 8.0 and acetic acid solution prior to PCR. RNA-protein fusion molecules were quantified by scintillation counting in the presence of scintillation fluid. Immunoprecipitation and recovery of cDNA from the bound complexes was accomplished as described above, except that [γ-³²P]ATP was added to the kinase reaction in round one of the cellular mRNA-protein fusion molecule selection to facilitate detection of the fusions. Repeated rounds of selection produced an enriched pool of v-abl cellular substrates.

Enrichment of cellular mRNA-protein fusions containing phosphorylated tyrosines

Approximately 1 pmole of the starting cellular mRNA-protein library was used in the first round of the proteomic selection. In order to minimize the loss of genetic diversity, the pre-clear step was omitted in the first round of the selection. The first two rounds of the selection did not yield detectable radioactive counts in the elutions, although subsequent PCR reactions yielded genetic material that was taken to the next round. In contrast, approximately 2% of the input mRNA-protein fusion molecules were eluted (fractions E1+E2) from the 4G10 antibody in round three, and this increased to 6% in rounds four and five. The percentage of input mRNA-protein fusion molecules eluted in rounds four and five was significantly greater than that in wash five (fraction W5) and was similar to that recovered using the optimal v-abl target. In addition, PCR analysis demonstrated that the last wash of round five (fraction W5) was devoid of mRNA-protein fusion molecules. Together these results demonstrate that substrates of v-abl were enriched from the cellular mRNA-protein library following five rounds of selection and PCR amplification of genetic material.

Analysis of selected pools

Since the starting cellular mRNA-protein fusion library consisted of the full repertoire of proteins expressed in human bone marrow cells, the enrichment of desired clones could be rapidly ascertained by hybridization to human cDNA microarrays. As a first step in the analysis of the selected pools, DNA from rounds four and five of the selection were hybridized to cDNA microarrays containing >18,000 EST clones. The hybridization was carried out as follows. The selected pools of PCR products were labeled by random priming using the Rediprime DNA labeling system (Amersham Pharmacia) and [α-³²P] dCTP (2000-4000 Ci/mmol). Unincorporated [α-³²P]-dCTP was removed by chromatography and labeled DNA was hybridized to the filters (GenomeSystems Inc., St. Louis, Mo.), as described by the manufacturer. Hybridization signals were visualized by phosphorimager analysis and the relative intensities were determined using the manufacturer's Genome Discovery Software (GDS).

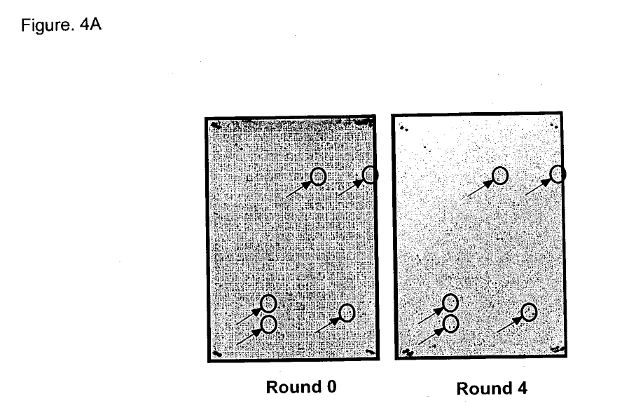

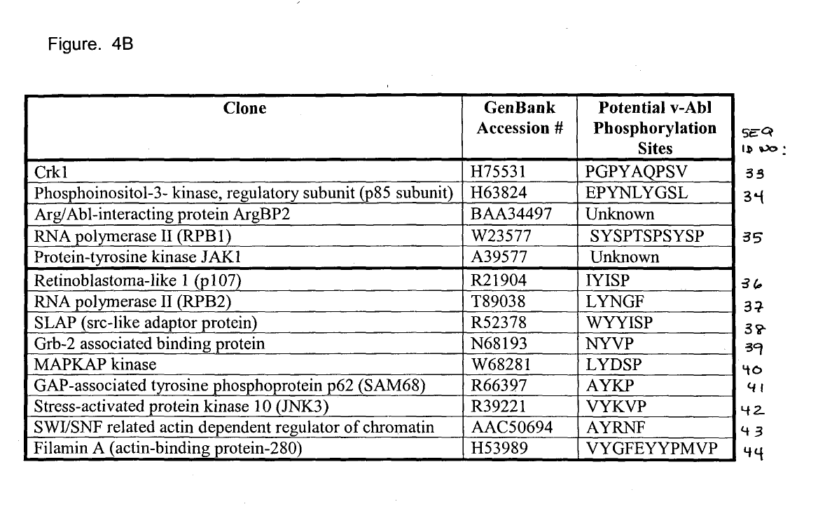

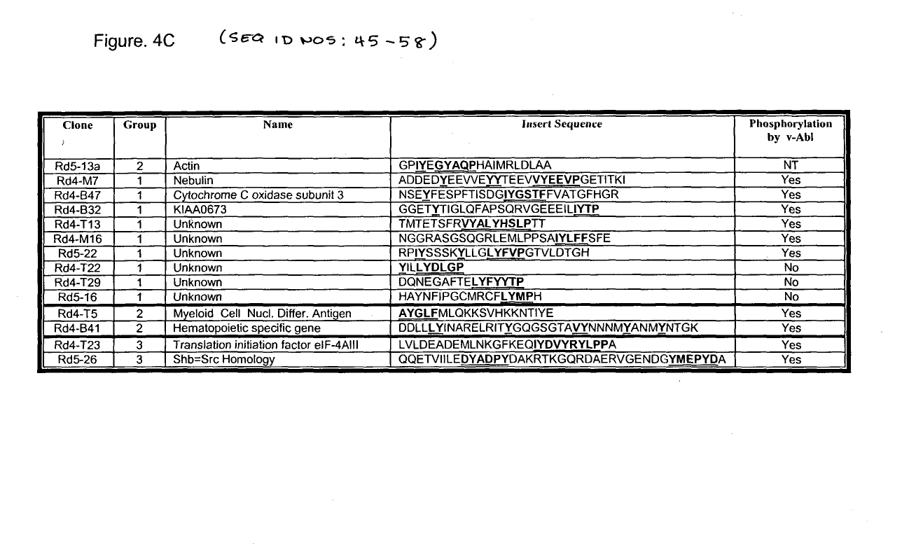

The hybridization intensities of the DNA obtained from rounds four and five were compared to those obtained with the starting (round zero) library (FIG. 4A). The filter contained four known v-abl substrates: Crkl (Feller et al, Trends Biochem. Sci. 19:453-458, 1994; Ren et al., Genes Dev. 8:783-795, 1994); the regulatory subunit (p85) of phosphoinositol-3-kinase (Varticovski et al., Mol. Cell Biol. 11:1107-1113, 1991); the Arg/Abl-interacting protein ArgBP2 (Wang et al., J. Biol. Chem. 272:17542-17550, 1997); and the JAK1 tyrosine kinase (Danial et al., Mol Cell Biol. 18:6795-804, 1998; and Danial and Rothman, Oncogene 19:2523-2531, 2000), as well as the large subunit of RNA polymerase II, a previously reported target of c-abl (Baskaran et al, Proc. Natl. Acad. Sci. U.S.A. 90:11167-11171, 1993). All of these targets were enriched during the selection procedure (FIG. 4B). Numerous other ESTs containing potential v-abl phosphorylation sites were also detected in the hybridization analysis. In most cases, these correspond to proteins known to interact with v-abl, or proteins that are involved in cellular processes affected by v-abl (Zou et al., J. Biol. Chem. 274:18141-18144, 1999; Laneuvill, Semin. Immunol. 7:255-266, 1995; Pendergast, Curr. Opin. Cell. Biol. 8:174-181, 1996).

The potential v-abl phosphorylation sites identified in the filter analysis were very similar to the v-abl consensus sequence identified from the selection of a randomized peptide library. Nevertheless, to examine the nature of the enriched pools more precisely, the pools from rounds four and five of the selection were subcloned and the resulting clones were sequenced. Sequence analysis of 102 clones indicated that the v-abl target clones could be divided into the following three major groups: 1) clones fitting the kinase consensus sequence I/L/V-Y-X₁₋₅-P/F (64/102); 2) those lacking a downstream proline/phenylalanine residue (14/102); and 3) those lacking an upstream hydrophobic residue (14/102). Only a small fraction of the clones (10/102) had more than one difference from the consensus sequence or no tyrosine.

To determine whether members of the three classes could serve as v-abl substrates, representative clones from each of the major groups were expressed individually as free peptides and tested for v-abl phosphorylation in vitro. As summarized in FIG. 4C, clones representing all three groups were phosphorylated. Only three of the 13 clones tested in the individual kinase assays were not phosphorylated by v-abl. These sequences closely matched the v-abl consensus sequence and failure to detect phosphorylation might be due to the close proximity of the tyrosines to the FLAG epitope and the antibody used to immunoprecipitate the free proteins. Together these results demonstrated that cellular substrates of v-abl could be selected from a cellular mRNA-protein fusion library and that the repertoire of potential v-abl targets was much broader than indicated by previous studies.

Homology searches using the sequenced clones identified 13 known genes and three predicted ORFs. One of the known genes was identified as actin, previously published as a v-abl interacting protein (Van Etten et al., J. Cell. Biol. 124:325-340, 1994; Wang, Curr. Opin. Genet. Dev. 3:35-43, 1993). The translation elongation factor elF-4AIII was found twice and the selected clone is phosphorylated by v-abl in vitro. Nebulin was found 31 times; and cytochrome C oxidase subunit 3 was found 25 times. Thirty-three clones showed no homology to any genes in the GenBank database.

Many of the nebulin and cytochrome C oxidase clones contained different 5′ or 3′ termini indicating that the clones arose from distinct priming events during construction of the library. This suggests that, as expected, overlapping protein domains are present in the mRNA-protein fusion library. Nebulin is closely associated with actin and may represent a novel substrate for v-abl given its high representation in the selected pools and its phosphorylation by v-abl in vitro. In contrast, the segment of the selected cytochrome C oxidase is embedded inside the core of the full-length protein and may not be accessible to v-abl in vivo.

### EXAMPLE 2

Shg Polypeptides and Nucleic Acids

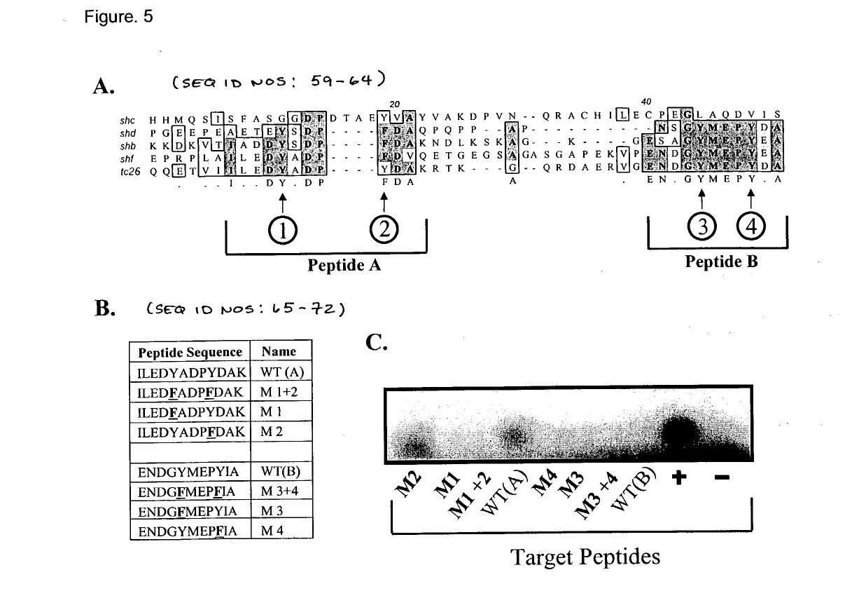

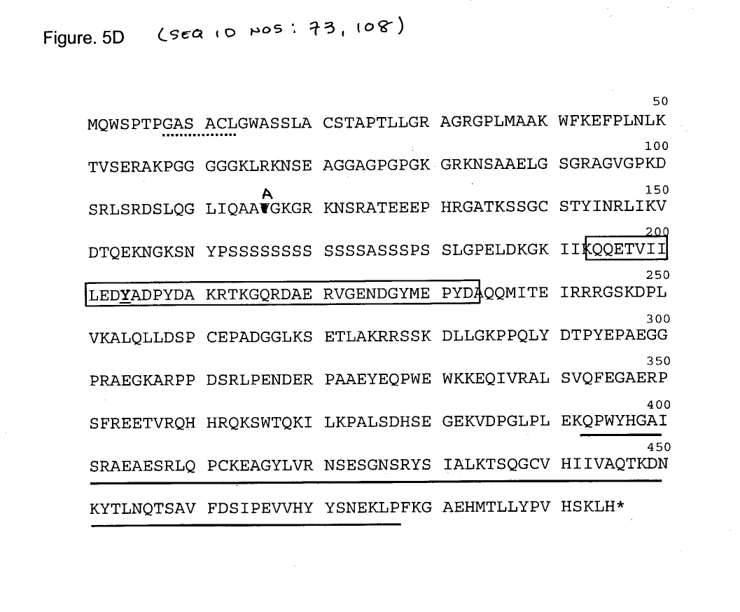

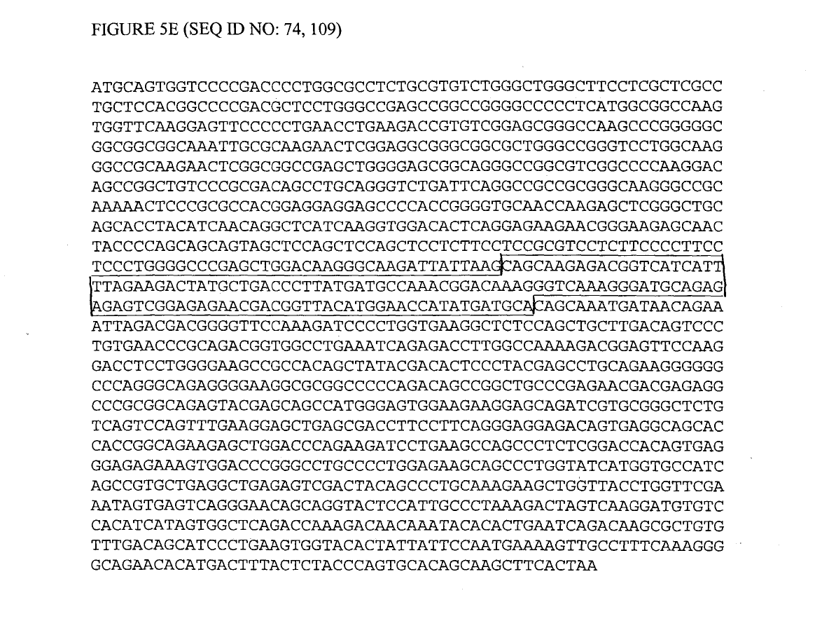

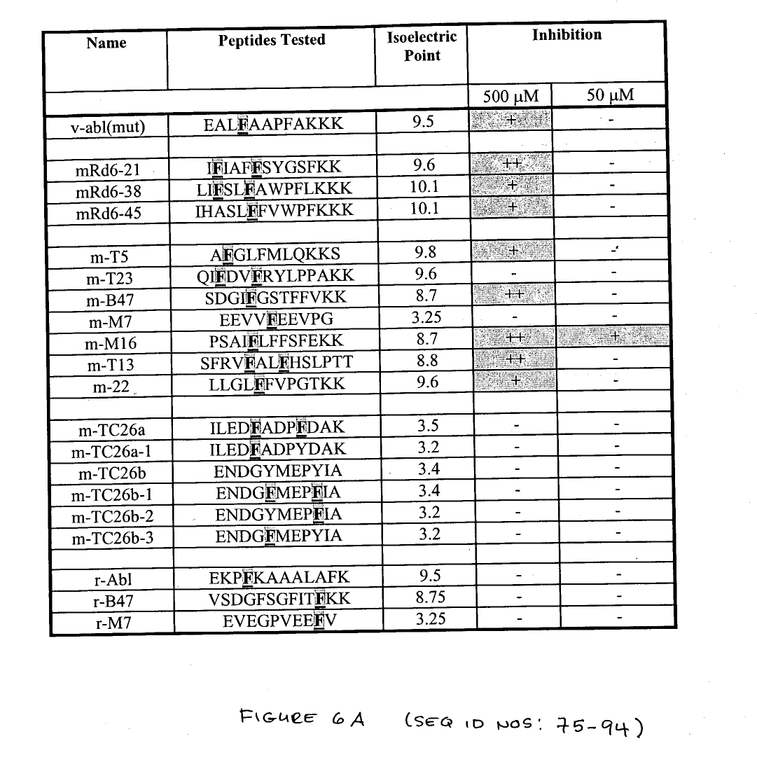

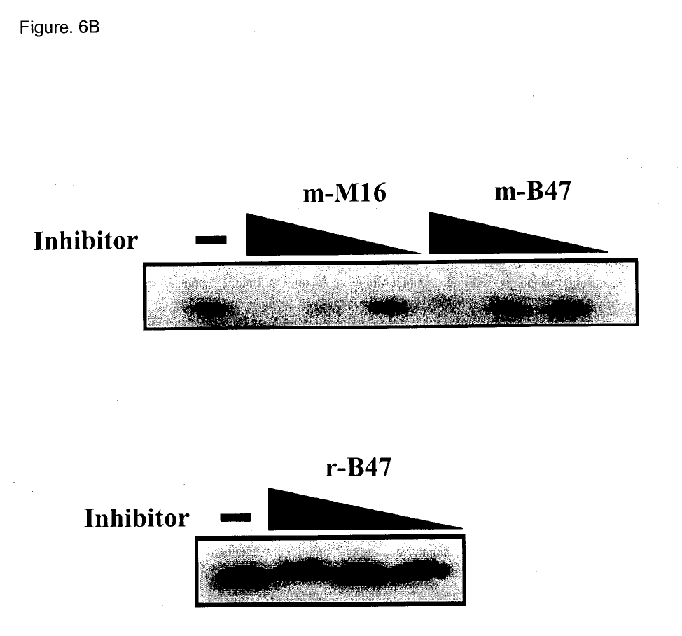

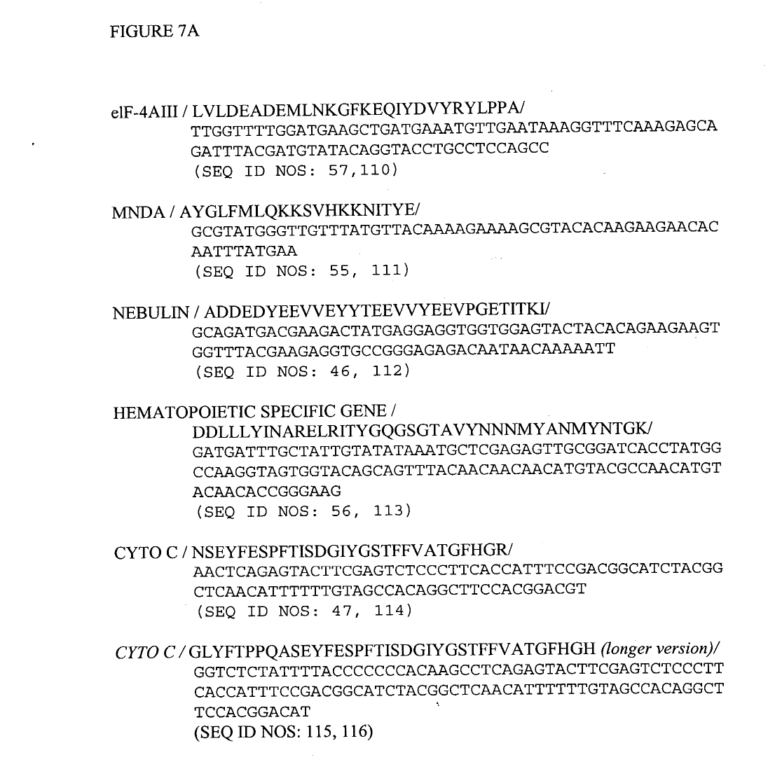

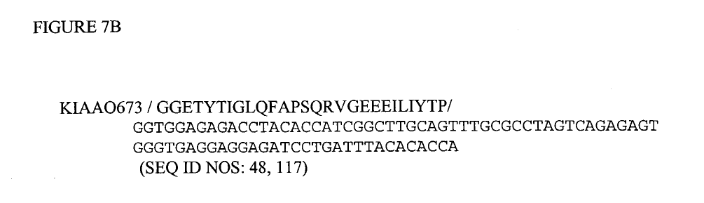

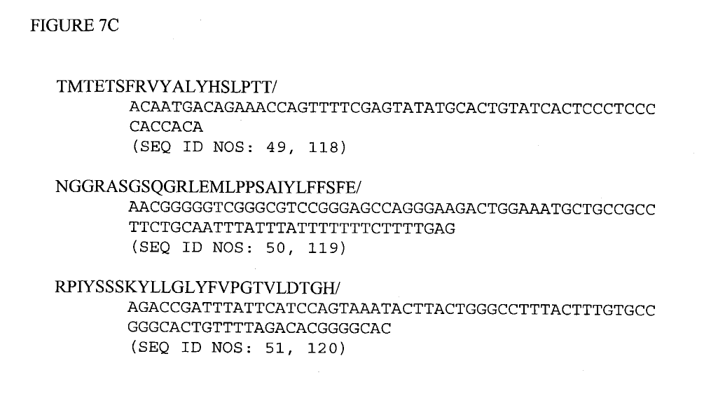

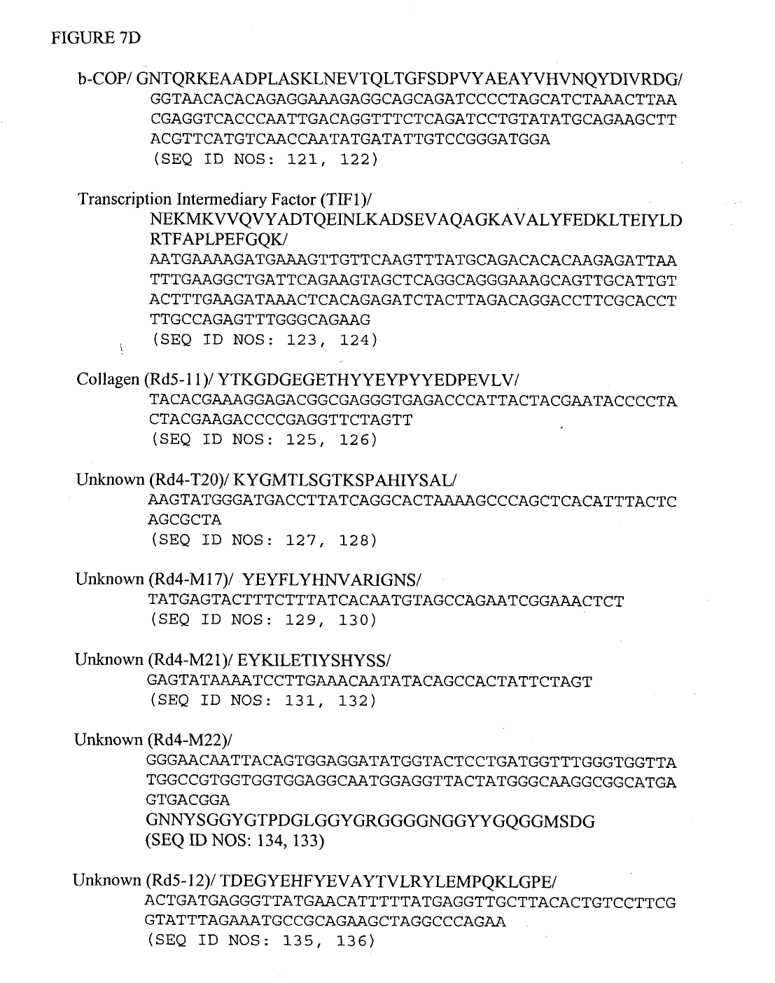

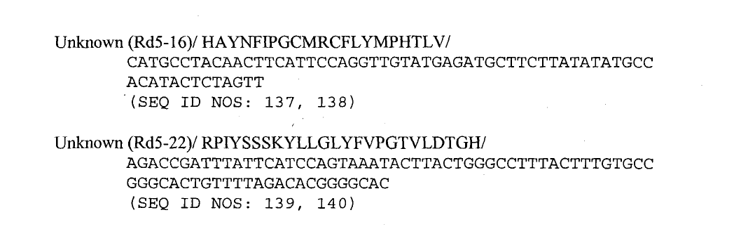

Sequence analysis indicated that one of the fragments (TC26) obtained in round five of the selection, had homology to a class of SH2-domain (Src homology 2)-containing adaptor proteins (FIG. 5A). Two members of this family, Shc and Shd, are phosphorylated by v-abl in vivo. TC26 contains four potential v-abl phosphorylation sites. Experiments using mutagenized peptides demonstrated that only the first tyrosine is a substrate for v-abl (FIGS. 5B and 5C). Because this family of proteins appears to be important for abl function, a PCR-based strategy was used to obtain sequence information for the full-length clone, termed shg (FIGS. 5D and 5E).

Full-length shg was cloned through a nested PCR strategy using gene-specific (TC26) and flanking adaptor primers to amplify the 3′ and partial 5′ shg sequences from double stranded cDNA prepared from human bone marrow mRNA (Marathon-Ready™ cDNA, Palo Alto, Calif.). Additional 5′ shg sequences were obtained by 5′ RACE (GibcoBRL Life Technologies) essentially as described by the manufacturer, except that cDNA synthesis was done at 55° C., and the terminal transferase reaction was done in the presence of dATP. Nested gene-specific primers and appropriate flanking primers (3′ RACE adaptor and AUAP primers, GibcoBRL Life Technologies) were used to obtain the terminal 5′ sequences of shg by PCR. PCR analysis using shg-specific flanking primers yielded a single 1.49 kbp band that hybridized to a transcript approximately 1.5 kbp in size following hybridization to Northern blots.

The Shg adaptor protein has a predicted molecular weight of 54 KD (495 aa) and is relatively basic (pI=9.2). Similar to other members of the family, Shg contains an SH2 domain near its carboxyl terminus (amino acids 393-477). The amino termini of all known family members are highly divergent, but Shg is unique in that it contains an N-terminal myristylation site (amino acids 8-13), which could serve to anchor this cytosolic protein to the cell membrane.

In additional experiments, it was demonstrated that the full-length Shg protein was phosphorylated by both v-abl and ZAP 70 in vitro, and that the full-length Shg protein, as well as the N-terminal 1-383 amino acids, were phosphorylated by Bcr-abl following overexpression in mammalian COS cells.

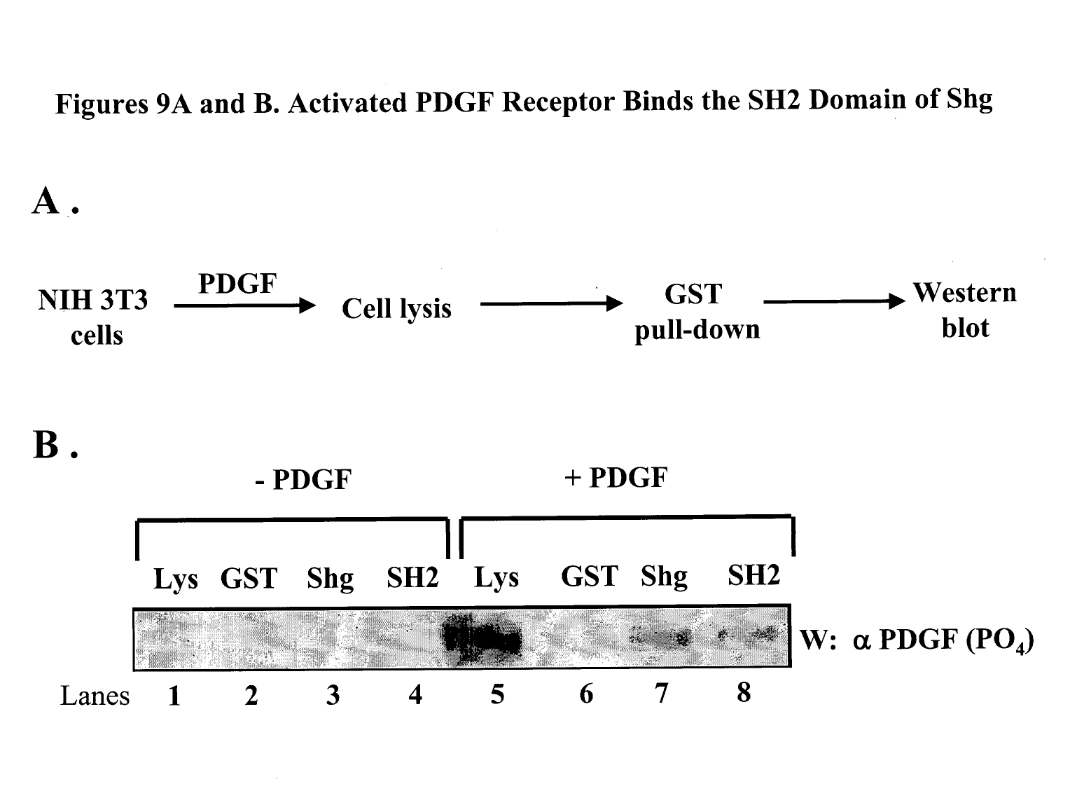

In further experiments, Shg was shown to contain a functional SH2 domain by demonstrating its interaction with activated platelet-derived growth factor (PDGF) receptor. In these experiments, NIH 3T3 cells were used as the source of the PDGF receptors. Receptors were activated by first incubating the cells (80% confluent, 100 mm dish) in Dulbecco's Modified Eagle Medium supplemented with 1% heat-inactivated calf serum and L-glutamine (0.3 mg/ml) for 4 hours under standard conditions. Cells were then washed twice with PBS, and incubated for another 4 hours in DMEM supplemented with 10 ng/ml of PDGF ligand (Upstate Biotechnology cat # 01-305) in the absence of serum. Unactivated cells serving as negative controls were treated as described above, except that the PDGF ligand was omitted from the growth medium. Following PDGF receptor activation, cells were washed twice with cold PBS and lysed in 3 ml of modified RIPA buffer (50 mM Tris, pH 7.4, 100 mM NaCl, 1 mM EDTA, 1% NP-40, 0.1 mM Na₃VO₄, 1 mM NaF, and a 1:1000 dilution of a protease inhibitor cocktail (Sigma, cat # P 8340)). Cell lysates were cleared by centrifugation at 12,000 rpm for 10 minutes at 4° C. Supernatants were incubated at 4° C. for 1 hour with either 2.7 nmoles of glutathione S-transferase (GST) protein (75 μL), 1.6 nmoles of ShgSH2-GST (100 μl) or 0.5 nmoles of Shg-GST fusion proteins (100 μl) attached to Glutathione Sepharose 4B beads. GST, and relevant fusion proteins, were over-expressed in E. coli and purified on Sepharose beads using standard procedures (Amersham Pharmacia Biotech). Following incubation of the lysates, complexes bound to GST or GST-fusion proteins were washed three times in modified RIPA buffer (above), two times in PBS and then resuspended in 50 μl of SDS-protein loading buffer (125 mM Tris, pH 6.8, 20% (v/v) glycerol, 4% SDS, 200 mM DTT, 0.2% bromophenol blue). Samples were boiled for 5 minutes and eluted proteins resolved by SDS-PAGE (4-12%). Proteins were transferred onto nitrocellulose paper and subjected to analysis by Western blotting using standard procedures. This set of experiments is represented schematically in FIG. 9A.

The ability of full length Shg (Shg) as well as its SH2 domain (SH2) to bind to activated PDGF receptors was assayed by probing the Western blots with a phospho-specific antibody against the PDGF receptor β (Y716) (Upstate Biotechnologies cat. # 07-021). As shown in FIG. 9B, incubation of cells with the PDGF ligand resulted in phosphorylation of the PDGF receptor (lane 5). GST fusion proteins containing full length Shg, or its SH2 domain alone (aa 383-496), bound to phosphorylated PDGF receptors in the cell lysates, as shown by the fact that the activated receptor could be detected by Western blotting using a phospho-PDGF receptor specific antibody (lanes 7 and 8). In contrast, activated PDGF receptors did not bind to the GST protein alone (lane 6). The interaction between Shg and the PDGF receptor is dependent upon phosphorylation of the receptor since no interaction was detected if the nonphosphorylated receptor was immunoprecipitated from cells that were not incubated with the PDGF ligand (lanes 2-4). These results provide further confirmation that the Shg polypeptide contains a functional SH2 domain.

In another set of experiments, K562 cell lines stably expressing FLAG-tagged Shg and its truncated derivatives were generated.

### EXAMPLE 3

Inhibition of Abl Kinase Activity

In addition to the information gained by the identification of novel kinase targets, common sequence motifs across selected targets allowed for a focused interrogation of potential active site inhibitors. The activity of several serine/threonine kinases has been inhibited using kinase pseudo-substrates in which the target serine has been mutated to an alanine (Cujec et al., Genes Dev. 11:2645-2657, 1997; Okamoto et al., Virology 270:337-344, 2000; Poteet-Smith et al., J. Biol. Chem. 272:379-388, 1997). In an analogous manner, the tyrosine kinase activity of ZAP-70 was inhibited by a pseudo-substrate containing a tyrosine to phenylalanine substitution mutation (Nishikawa et al., Mol. Cell 6:969-974, 2000).

Consequently, we sought to determine whether v-abl substrates selected from the random peptide and proteomic libraries could inhibit kinase activity if the target tyrosine was mutated to phenylalanine. To carry out these experiments, test peptides were synthetically generated that included a phenylalanine rather than a tyrosine at the phosphorylation site. Kinase inhibition assays contained 5 μM of the commercial v-abl substrate (EAIYAAPFAKKK; New England Biolabs) and increasing concentrations (5 μM, 50 μM, or 500 μM) of the test peptides. Peptides mRd6-21, mRd6-38, mRd6-45, mRd4-T5, Rd4-T23, Rd4-B47, rRd4-B47, Rd4-M16, and the R5-TC26b series were dissolved in a DMF (50%)-water (50%) solution (500 μM). The remaining peptides were soluble in water.

The results indicated that one of the mutagenized peptides (m-M16) inhibited v-abl activity at 50 μM (FIGS. 6A and 6B), while three additional peptides (mRd6-21, m-B47, and mT13) were effective inhibitors at concentrations of 500 μM. In marked contrast, a phenylalanine substitution in an optimized v-abl target sequence derived from selections of a randomized peptide library (v-abl-mut; Songyang et al., Nature 373:536-539, 1995) was less effective as a kinase inhibitor; further underlying the observation that distal amino acids are important determinants of catalytic specificity. A subset of control peptides (r-series) containing the same amino acids as the test peptides, but in a scrambled order, did not have a significant effect on v-abl kinase activity.

### EXAMPLE 4

Method for the Identification of Kinase Inhibitors

The present techniques enable a novel approach for the identification of kinase inhibitors. This approach begins with a library of candidate encoding nucleic acid sequences. The library may include species having a codon for an invariant amino acid, for example, an invariant phenylalanine embedded in a region encoding a series of randomized amino acids (such as that described above), or it may be a random and/or synthetic library or cellular library. The library, once synthesized, is used to generate nucleic acid-protein fusions, for example, as described in Roberts and Szostak (Proc. Natl. Acad. Sci. U.S.A. 94:12297-302, 1997) and Szostak et al. (WO 98/31700; and U.S. Pat. No. 6,261,553 B1), hereby incorporated by reference. The nucleic acid-protein library is incubated with a purified kinase of interest, and nucleic acid-protein fusions binding to the kinase are separated from unbound fusions. This separation step may be carried out by any standard technique, including any technique by which the kinase is bound to a solid support (such as a column or chip) or preferably by separating the kinase complexes based on a kinase-associated tag (for example, a flag-tag incorporated into the kinase sequence) or kinase-associated label (such as biotin). The nucleic acid portions of the one or more bound fusions are then amplified (for example, by PCR), and the selection and amplification procedure is repeated for several rounds to achieve a desired level of enrichment for a population of proteins that can bind to the kinase of interest. Selected proteins are then tested, either in the form of fusion proteins or purified or partially purified recombinant proteins, for their ability to inhibit kinase activity using any standard kinase assay. In these assays, the kinase, its substrate (preferably, its optimal substrate), and the inhibitory protein are incubated together and scored for a decreased ability of the kinase to phosphorylate its substrate in the presence of the inhibitor.

This technique may be used to identify inhibitors of any kinase, including without limitation, any tyrosine kinase or serine/threonine kinase.

### EXAMPLE 5

Serine/Threonine Kinase Targets

Serine/threonine kinase targets may also be selected by the mRNA display technique described above. To illustrate the workability of this technique, a known substrate of cAMP-dependent protein kinase (PKA) was detected as follows.

Standard kinase reactions containing the catalytic subunit of cAMP-dependent kinase and adenosine-5′-O-(3-thio) triphosphate as the phosphate donor were supplemented with the optimized substrate peptide (GRTGRRNSI) (SEQ ID NO: 141). Radiolabeled [γ-³⁵S]ATP was added to the reactions as a tracer. Reactions were incubated at 30° C. for 1 hour. Free [γ-³⁵S]ATP was then separated from the peptide by filtration through a G25 spin column. Transfer of the γ-phosphate to the peptide was monitored by spotting a fraction of the reaction onto Whatman P81 filter paper and washing with 75 mM phosphoric acid. Phosphorylated peptides were incubated with streptavidin beads in the presence or absence of bromo-benzyl biotin at 25° C. for 1 hour. Beads were washed extensively with modified RIPA buffer (50 mM TRIS pH 7.4, 1% Nonidet P40, 150 mM NaCl, 1 mM EGTA, 10% glycerol), and the amount of peptide bound to the beads was quantified by scintillation counting.

The bromo-benzyl biotin reagent was generated as follows. 4-bromomethylphenylacetic acid (Aldrich) was made to react with the amine group of NH₂-PEO-LC-biotin (Pierce) by incubating the chemicals in a solution of dimethylformamide (DMF) at room temperature in the presence of dicyclohexylcarbodiimide.

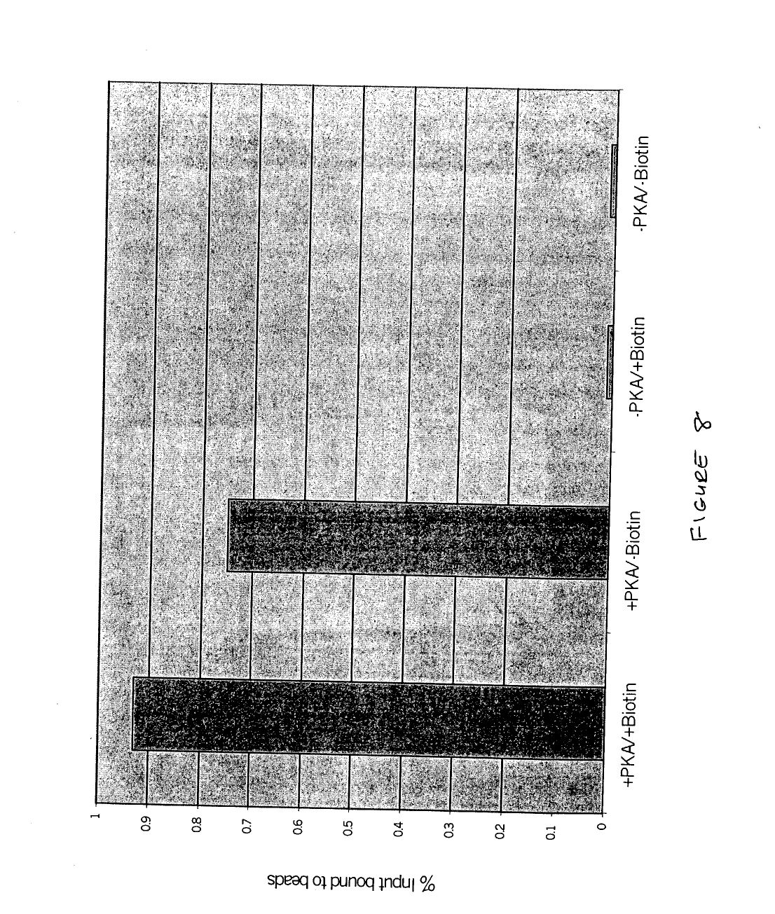

The results of this experiment are shown in FIG. 8.

### EXAMPLE 6

Kinase Target and Inhibitor Polypeptides, Nucleic acids, and Related Molecules

Identification of a kinase target, such as the Shg protein, or a kinase inhibitor (e.g., those inhibitors described herein) facilitates the production of related molecules, such as related genes from other species, gene fragments and analogs, and antibodies, as well as transgenic animals. Exemplary methods for producing these related embodiments are described below.

Cloning full length nucleic acid molecules encoding kinase target polypeptides

Nucleic acid molecules encoding the full length polypeptide sequences of any identified kinase target polypeptide can readily be cloned using standard hybridization or PCR cloning techniques and DNA from its source, for example, as described in Ausubel et al. (supra). An exemplary method for obtaining the full length polypeptide sequences employs a standard nested PCR strategy that can be used with gene-specific (obtained from the nucleic acid sequence encoding the kinase target polypeptide) and flanking adaptors from double stranded cDNA prepared from the source of the identified kinase target polypeptide. In addition, 5′ flanking sequence can be obtained using 5′ RACE techniques known to those of skill in the art.

Synthesis of kinase target or inhibitor polypeptides

Additional characteristics of kinase target or inhibitor polypeptides may be analyzed by synthesizing the polypeptides in various cell types or in vitro systems. The function of these polypeptides may then be examined under different physiological conditions. Alternatively, cell lines may be produced which overexpress a nucleic acid encoding the kinase target or inhibitor polypeptide, allowing purification of the polypeptide for biochemical characterization, large-scale production, antibody production, or patient therapy.

For polypeptide expression, eukaryotic and prokaryotic expression systems may be generated in which nucleic acid sequences encoding kinase target or inhibitor polypeptides are introduced into a plasmid or other vector, which is then used to transform living cells. Constructs in which the nucleic acid sequences are inserted in the correct orientation into an expression plasmid may be used for protein expression. Alternatively, portions of gene sequences encoding the kinase target or inhibitor polypeptide, including wild-type or mutant polypeptide sequences, may be inserted. Prokaryotic and eukaryotic expression systems allow various important functional domains of the kinase target or inhibitor polypeptides to be recovered, if desired, as fusion proteins, and then used for binding, structural, and functional studies and also for the generation of appropriate antibodies. If desired, the polypeptide may be expressed under the control of an inducible promoter in those cells.

Standard expression vectors contain promoters that direct the synthesis of large amounts of mRNA corresponding to the inserted nucleic acid encoding a kinase target or inhibitor polypeptide in the plasmid-bearing cells. They may also include eukaryotic or prokaryotic origin of replication sequences allowing for their autonomous replication within the host organism, sequences that encode genetic traits that allow vector-containing cells to be selected in the presence of otherwise toxic drugs, and sequences that increase the efficiency with which the synthesized mRNA is translated. Cell lines may also be produced that have integrated the vector into the genomic DNA, and in this manner the gene product is produced on a continuous basis.

Expression of foreign sequences in bacteria such as E. coli requires the insertion of the nucleic acid sequence encoding the kinase target or inhibitor polypeptide into a bacterial expression vector. Such plasmid vectors contain several elements required for the propagation of the plasmid in bacteria, and for expression of the DNA inserted into the plasmid. Propagation of only plasmid-bearing bacteria is achieved by introducing, into the plasmid, selectable marker-encoding sequences that allow plasmid-bearing bacteria to grow in the presence of otherwise toxic drugs. The plasmid also contains a transcriptional promoter capable of producing large amounts of mRNA from the cloned gene. Such promoters may be (but are not necessarily) inducible promoters that initiate transcription upon induction. The plasmid also preferably contains a polylinker to simplify insertion of the gene in the correct orientation within the vector.

Once the appropriate expression vectors containing a nucleic acid sequence encoding a kinase target or inhibitor polypeptide, or fragment, fusion, or mutant thereof, are constructed, they are introduced into an appropriate host cell by transformation techniques, including, e.g., calcium phosphate transfection, DEAE-dextran transfection, electroporation, microinjection, protoplast fusion, and liposome-mediated transfection. The host cells that are transfected with the vectors of this invention may include (but are not limited to) E. coli or other bacteria, yeast, fungi, insect cells (using, for example, baculoviral vectors for expression), or cells derived from mice, humans, or other animals. Mammalian cells can also be used to express kinase target or inhibitor polypeptides using, for example, a vaccinia virus expression system described, for example, in Ausubel et al. (supra).

Expression of kinase target or inhibitor polypeptides, fusions, polypeptide fragments, or mutants encoded by cloned DNA is also possible using, for example, the T7 late-promoter expression system. This system depends on the regulated expression of T7 RNA polymerase, an enzyme encoded in the DNA of bacteriophage T7. The T7 RNA polymerase initiates transcription at a specific 23-bp promoter sequence called the T7 late promoter. Copies of the T7 late promoter are located at several sites on the T7 genome, but none is present in E. coli chromosomal DNA. As a result, in T7-infected E. coli cells, T7 RNA polymerase catalyzes transcription of viral genes but not of E. coli genes. In this expression system, recombinant E. coli cells are first engineered to carry the gene encoding T7 RNA polymerase next to the lac promoter. In the presence of IPTG, these cells transcribe the T7 polymerase gene at a high rate and synthesize abundant amounts of T7 RNA polymerase. These cells are then transformed with plasmid vectors that carry a copy of the T7 late promoter protein. When IPTG is added to the culture medium containing these transformed E. coli cells, large amounts of T7 RNA polymerase are produced. The polymerase then binds to the T7 late promoter on the plasmid expression vectors, catalyzing transcription of the inserted cDNA at a high rate. Since each E. coli cell contains many copies of the expression vector, large amounts of mRNA corresponding to the cloned cDNA can be produced in this system. The resulting protein can be radioactively labeled. Plasmid vectors containing late promoters and the corresponding RNA polymerases from related bacteriophages such as T3, T5, and SP6 may also be used for production of proteins from cloned DNA. E. coli can also be used for expression using an M13 phage such as mGPI-2. Furthermore, vectors that contain phage lambda regulatory sequences, or vectors that direct the expression of fusion proteins, for example, a maltose-binding protein fusion protein or a glutathione-S-transferase fusion protein, also may be used for expression in E. coli.

Eukaryotic expression systems are useful for obtaining appropriate post-translational modification of expressed polypeptides. Transient transfection of a eukaryotic expression plasmid allows the transient production of kinase target polypeptides by a transfected host cell. Kinase target polypeptides may also be produced by a stably-transfected mammalian cell line. A number of vectors suitable for stable transfection of mammalian cells are available to the public (e.g., see Pouwels et al., Cloning Vectors: A Laboratory Manual, 1985, Supp. 1987), as are methods for constructing such cell lines (see e.g., Ausubel et al., supra). In one example, a nucleic acid molecule encoding a kinase target or inhibitor polypeptide, fusion, mutant, or polypeptide fragment is cloned into an expression vector that includes the dihydrofolate reductase (DHFR) gene. Integration of the plasmid and, therefore, integration of the nucleic acid sequence encoding the abl kinase target polypeptide into the host cell chromosome is selected for by inclusion of 0.01-300 μM methotrexate in the cell culture medium (as described, for example, in Ausubel et al., supra). This dominant selection can be accomplished in most cell types. Recombinant protein expression can be increased by DHFR-mediated amplification of the transfected gene. Methods for selecting cell lines bearing gene amplifications are described in Ausubel et al. (supra). These methods generally involve extended culture in medium containing gradually increasing levels of methotrexate. The most commonly used DHFR-containing expression vectors are pCVSEII-DHFR and pAdD26SV(A) (described, for example, in Ausubel et al., supra). The host cells described above or, preferably, a DHFR-deficient CHO cell line (e.g., CHO DHFR⁻ cells, ATCC Accession No. CRL 9096) are among those most preferred for DHFR selection of a stably-transfected cell line or DHFR-mediated gene amplification.

Eukaryotic cell expression of kinase target or inhibitor polypeptides facilitates studies of the gene and gene products encoding these polypeptides, including determination of proper expression and post-translational modifications for biological activity, identifying regulatory elements located in the 5′, 3′, and intron regions of their nucleic acid molecules. It also permits the production of large amounts of normal and mutant proteins for isolation and purification, and the use of cells expressing the kinase target polypeptides as a functional assay system for antibodies generated against the protein. Eukaryotic cells expressing kinase target polypeptides may also be used to test the effectiveness of pharmacological agents on kinase-related disorders (for example, for the abl kinase, screens for drugs to treat cell proliferative diseases). Expression of kinase target polypeptides, fusions, mutants, and polypeptide fragments in eukaryotic cells also enables the study of the function of the normal complete polypeptide, specific portions of the polypeptide, or of naturally occurring polymorphisms and artificially-produced mutated polypeptides.

Once the recombinant protein is expressed, it can be isolated from the expressing cells by cell lysis followed by protein purification techniques, such as affinity chromatography. In this example, a specific antibody, which may be produced by the methods described herein, can be attached to a column and used to isolate the recombinant kinase target or inhibitor polypeptides. Lysis and fractionation of polypeptide-harboring cells prior to affinity chromatography may be performed by standard methods (see e.g., Ausubel et al. (supra). Once isolated, the recombinant protein can, if desired, be purified further, e.g., by high performance liquid chromatography (HPLC; e.g., see Fisher, Laboratory Techniques In Biochemistry And Molecular Biology, Work and Burdon, Eds., Elsevier, 1980).

Polypeptides of the invention, particularly short kinase target or inhibitor fragments, can also be produced by chemical synthesis (e.g., by the methods described in Solid Phase Peptide Synthesis, 2nd ed., 1984, The Pierce Chemical Co., Rockford, Ill.). These general techniques of polypeptide expression and purification can also be used to produce and isolate useful kinase target or inhibitor polypeptide fragments or analogs, as described herein.

In addition, prokaryotic and eukaryotic in vitro systems can be utilized for the generation of kinase target or inhibitor polypeptides. Such methods are described, for example, by Ausubel et al.(supra). Proteins can be synthesized using, for example, in vitro transcription and translation methods. Rabbit reticulocyte lysates, wheat germ lysates, or E. coli lysates can be used to translate exogenous mRNAs from a variety of eukaryotic and prokaryotic sources. Kits for the in vitro production of polypeptides are available, for example, from Ambion (Austin, Tex.).

Kinase target-specific antibodies

In order to prepare polyclonal antibodies, kinase target polypeptides, fragments of kinase target polypeptides, or fusion polypeptides containing defined portions of kinase target polypeptides may be synthesized in bacteria by expression of corresponding DNA sequences in a suitable cloning vehicle. Fusion proteins are commonly used as a source of antigen for producing antibodies. Two widely used expression systems for E. coli are lacZ fusions using the pUR series of vectors and trpE fusions using the pATH vectors. The proteins can be purified, and then coupled to a carrier protein and mixed with Freund's adjuvant (to enhance stimulation of the antigenic response in an innoculated animal) and injected into rabbits or other laboratory animals. Alternatively, protein can be isolated from kinase target polypeptide-expressing cultured cells. Following booster injections at bi-weekly intervals, the rabbits or other laboratory animals are then bled and the sera isolated. The sera can be used directly or can be purified prior to use by various methods, including affinity chromatography employing reagents such as Protein A-Sepharose, antigen-Sepharose, and anti-mouse-Ig-Sepharose. The sera can then be used to probe protein extracts from kinase target polypeptide-expressing tissue electrophoretically fractionated on a polyacrylamide gel to identify kinase target polypeptides. Alternatively, synthetic peptides can be made that correspond to the antigenic portions of the protein and used to innoculate the animals.

In order to generate a peptide for use in making, for example, abl kinase target polypeptide-specific antibodies, an abl kinase target polypeptide sequence may be expressed as a C-terminal fusion with glutathione S-transferase (GST; Smith et al., Gene 67:31-40, 1988). The fusion protein may be purified on glutathione-Sepharose beads, eluted with glutathione, cleaved with thrombin (at the engineered cleavage site), and purified to the degree required to successfully immunize rabbits. Primary immunizations may be carried out with Freund's complete adjuvant and subsequent immunizations performed with Freund's incomplete adjuvant. Antibody titers are monitored by Western blot and immunoprecipitation analyses using the thrombin-cleaved abl kinase target polypeptide fragment of the abl kinase target-GST fusion polypeptide. Immune sera are affinity purified using CNBr-Sepharose-coupled abl kinase target polypeptide. Antiserum specificity may be determined using a panel of unrelated GST fusion proteins.

Alternatively, monoclonal antibodies that recognize kinase target polypeptides may also be produced by using, as an antigen, a kinase target polypeptide isolated from kinase target polypeptide-expressing cultured cells or kinase target polypeptide isolated from tissues. The cell extracts, or recombinant protein extracts containing kinase target polypeptide, may, for example, be injected with Freund's adjuvant into mice. Several days after being injected, the mouse spleens are removed, the tissues are disaggregated, and the spleen cells are suspended in phosphate buffered saline (PBS). The spleen cells serve as a source of lymphocytes, some of which are producing antibody of the appropriate specificity. These are then fused with permanently growing myeloma partner cells, and the products of the fusion are plated into a number of tissue culture wells in the presence of a selective agent such as hypoxanthine, aminopterine, and thymidine (HAT). The wells are then screened by ELISA to identify those containing cells making antibody capable of binding a kinase target polypeptide or polypeptide fragment or mutant thereof. These are then re-plated and after a period of growth, these wells are again screened to identify antibody-producing cells. Several cloning procedures are carried out until over 90% of the wells contain single clones that are positive for antibody production. From this procedure a stable line of clones that produce the antibody is established. The monoclonal antibody can then be purified by affinity chromatography using Protein A Sepharose, ion-exchange chromatography, as well as variations and combinations of these techniques. Truncated versions of monoclonal antibodies may also be produced by recombinant methods in which plasmids are generated that express the desired monoclonal antibody fragment(s) in a suitable host.

As an alternate or adjunct immunogen to GST fusion proteins, peptides corresponding to relatively unique hydrophilic regions of kinase target polypeptide may be generated and coupled to keyhole limpet hemocyanin (KLH) through an introduced C-terminal lysine. Antiserum to each of these peptides is similarly affinity-purified on peptides conjugated to BSA, and specificity is tested by ELISA and Western blotting using peptide conjugates, and by Western blotting and immunoprecipitation using kinase target polypeptide, for example, expressed as a GST fusion protein.

Alternatively, monoclonal antibodies may be prepared using the kinase target binding polypeptides described above and standard hybridoma technology (see, e.g., Kohler et al., Nature 256:495, 1975; Kohler et al., Eur. J. Immunol. 6:511, 1976; Kohler et al., Eur. J. Immunol. 6:292, 1976; Hammerling et al., In Monoclonal Antibodies and T Cell Hybridomas, Elsevier, New York, N.Y., 1981; and Ausubel et al. (supra)). Once produced, monoclonal antibodies are also tested for specific kinase target polypeptide recognition by Western blot or immunoprecipitation analysis (by the methods described in Ausubel et al., supra).

Monoclonal and polyclonal antibodies that specifically recognize a kinase target polypeptide (or fragments thereof), such as those described herein, are considered useful in the invention. Antibodies that inhibit the activity of a kinase target polypeptide described herein may be especially useful in preventing or slowing the development of a disease caused by inappropriate expression of a wild type or mutant kinase target polypeptide.

Antibodies of the invention may be produced using kinase target amino acid sequences that do not reside within highly conserved regions, and that appear likely to be antigenic, as analyzed by criteria such as those provided by the Peptide Structure Program (Genetics Computer Group Sequence Analysis Package, Program Manual for the GCG Package, Version 7, 1991) using the algorithm of Jameson and Wolf (CABIOS 4:181, 1988). These fragments can be generated by standard techniques, e.g., by PCR, and cloned into the pGEX expression vector (Ausubel et al., supra). GST fusion proteins are expressed in E. coli and purified using a glutathione-agarose affinity matrix as described in Ausubel et al., supra). To generate rabbit polyclonal antibodies, and to minimize the potential for obtaining antisera that is non-specific, or exhibits low-affinity binding to a kinase target polypeptide, two or three fusions are generated for each protein, and each fusion is injected into at least two rabbits. Antisera are raised by injections in series, preferably including at least three booster injections.

In addition to intact monoclonal and polyclonal anti-kinase target polypeptide antibodies, the invention features various genetically engineered antibodies, humanized antibodies, and antibody fragments, including F(ab′)2, Fab′, Fab, Fv, and sFv fragments. Antibodies can be humanized by methods known in the art, e.g., monoclonal antibodies with a desired binding specificity can be commercially humanized (Scotgene, Scotland; Oxford Molecular, Palo Alto, Calif.). Fully human antibodies, such as those expressed in transgenic animals, are also features of the invention (Green et al., Nature Genetics 7:13-21, 1994).

Ladner (U.S. Pat. No. 4,946,778 and 4,704,692) describes methods for preparing single polypeptide chain antibodies. Ward et al. (Nature 341:544-546, 1989) describe the preparation of heavy chain variable domains, which they term single domain antibodies, that have high antigen-binding affinities. McCafferty et al. (Nature 348:552-554, 1990) show that complete antibody V domains can be displayed on the surface of fd bacteriophage, that the phage bind specifically to antigen, and that rare phage (one in a million) can be isolated after affinity chromatography. Boss et al. (U.S. Pat. No. 4,816,397) describe various methods for producing immunoglobulins, and immunologically functional fragments thereof, which include at least the variable domains of the heavy and light chain in a single host cell. Cabilly et al. (U.S. Pat. No. 4,816,567) describe methods for preparing chimeric antibodies.

Affinity reagents or polypeptides from randomized polypeptide libraries that bind tightly to a desired polypeptides, for example, kinase target polypeptides, fragments of kinase target polypeptides, or fusion polypeptides containing defined portions of kinase target polypeptides can also be obtained, using methods known to one skilled in the art. In addition, polypeptide affinity scaffolds may be used to bind a polypeptide of interest or to identify or optimize a polypeptide that binds to a polypeptide of interest, for example, kinase target polypeptides, fragments of kinase target polypeptides, or fusion polypeptides containing defined portions of kinase target polypeptides. Such methods are described, for example, by Lipovsek et al. (WO 00/34784), hereby incorporated by reference.

Construction of transgenic animals and knockout animals

Characterization of kinase target polypeptide genes provides information that allows kinase target polypeptide knockout animal models to be developed by homologous recombination. Similarly, animal models of kinase target polypeptide overproduction may be generated by integrating one or more kinase target polypeptide sequences into the genome, according to standard transgenic techniques. Moreover, the effect of kinase target polypeptide gene mutations (e.g., dominant gene mutations) may be studied using transgenic mice carrying mutated kinase target polypeptide transgenes or by introducing such mutations into the endogenous kinase target polypeptide gene, using standard homologous recombination techniques.

Kinase target polypeptide knockout mice provide a tool for studying the role of a kinase target polypeptide in embryonic development and in disease. Moreover, such mice provide the means, in vivo, for testing therapeutic compounds (for example, kinase inhibitor polypeptides) for amelioration of diseases or conditions involving a kinase target polypeptide-dependent or kinase target polypeptide-affected pathway.

Construction of polypeptide knockout or overexpressing cell lines

Characterization of kinase target polypeptide genes also allows kinase target polypeptide cell culture models to be developed, in which the kinase target polypeptide is expressed or functions at a lower level than its wild-type counterpart cell. Such cell lines can be developed using standard antisense technologies. Similarly, cell culture models of kinase target polypeptide overproduction or overactivation may be generated by integrating one or more kinase target polypeptide sequences into the genome, according to standard molecular biology techniques. Moreover, the effect of kinase target polypeptide gene mutations (e.g., dominant gene mutations) may be studied using cell cultures model in which the cells contain and overexpress a mutated kinase target polypeptide.

Kinase target polypeptide knockout cells provide a tool for studying the role of kinase target polypeptide in cellular events, including cell proliferation. Moreover, such cell lines provide the cell culture means, for testing therapeutic compounds for modulation of kinase activity, or cell death, proliferation, or differentiation pathways. Compounds that modulate kinase activity, or cell death, proliferation, or differentiation pathways in these cell models can then be tested in animal models of diseases or conditions involving kinase activity.

Other Embodiments

In other embodiments, the invention includes any polypeptide that is substantially identical to a kinase target or inhibitor polypeptide (for example, an abl kinase target or inhibitor polypeptide); such homologues include other substantially pure naturally-occurring kinase target or inhibitor polypeptides as well as natural mutants; induced mutants; DNA sequences that encode polypeptides and also hybridize to the nucleic acid sequence encoding a kinase target or inhibitor polypeptides described herein under high stringency conditions or, less preferably under low stringency conditions (e.g., washing at 2×SSC at 40° C. with a probe length of at least 40 nucleotides); and proteins specifically bound by antisera directed to a kinase target or inhibitor polypeptide. The invention also includes chimeric polypeptides that include a kinase target or inhibitor polypeptide portion.

The invention further includes analogs of any naturally-occurring kinase target or inhibitor polypeptide. Analogs can differ from the naturally-occurring kinase target or inhibitor polypeptide by amino acid sequence differences, by post-translational modifications, or by both. Analogs of the invention will generally exhibit at least 85%, more preferably, 90%, and most preferably, 95% or even 99% identity with all or part of a naturally-occurring kinase target or inhibitor polypeptide sequence. The length of sequence comparison is at least 5 amino acid residues, preferably, at least 10 amino acid residues, and more preferably, the full length of the polypeptide sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring kinase target or inhibitor polypeptide by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs that contain residues other than L-amino acids, e.g., D-amino acids or non-naturally occurring or synthetic amino acids, e.g., β or γ amino acids.

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the appended claims.

Other embodiments are within the claims.

What is claimed is:

1. A substantially pure Shg polypeptide.

2. The substantially pure polypeptide of claim 1, wherein said polypeptide comprises the amino acid sequence of SEQ ID NO: 73.

3. An isolated nucleic acid molecule encoding the Shg polypeptide of claim 1.

4. The isolated nucleic acid molecule of claim 3, wherein said nucleic acid molecule comprises SEQ ID NO: 74.

5. A vector comprising the isolated nucleic acid molecule of claim 3.

6. A cell comprising the isolated nucleic acid molecule of claim 3 or the vector of claim 5.

7. A cell comprising the vector of claim 5.

8. A substantially pure polypeptide that is phosphorylated by an abl kinase, said polypeptide comprising the sequence of any of SEQ ID NOS: 49-51, 127, 129, 131, 133, 135, 137, and 139.

9. An isolated nucleic acid molecule encoding a polypeptide of claim 8.

10. The isolated nucleic acid of claim 9, wherein said nucleic acid comprises the sequence of any of SEQ ID NOS: 118-120, 128, 130, 132, 134, 136, 138, and 140.

11. A substantially pure polypeptide that inhibits the activity of a kinase, said polypeptide comprising the sequence of any of SEQ ID NOS: 76-79, 81, and 83-85.

12. An isolated nucleic acid molecule encoding a polypeptide of claim 11.

13. A method of inhibiting abl kinase activity, said method comprising contacting said kinase with the polypeptide of claim 11.

14. The method of claim 13, wherein said method is carried out in a cell.

15. A method for identifying a protein phosphorylated by a kinase, and its coding sequence, said method comprising the steps of:
(a) contacting a population of nucleic acid-protein fusions with a kinase under conditions that allow phosphorylation of the protein portion of said nucleic acid-protein fusion by said kinase;
(b) separating phosphorylated nucleic acid-protein fusions from nonphosphorylated nucleic acid-protein fusions;
(c) amplifying the nucleic acid portions of said phosphorylated nucleic acid-protein fusions; and
(d) repeating steps (a)-(c) one or more times, using the amplified nucleic acid of step (c) to generate an enriched population of nucleic acid-protein fusions for use in step (a), thereby identifying a protein phosphorylated by a kinase, and its coding sequence.

16. The method of claim 15, wherein, in step (b), said phosphorylated nucleic acid-protein fusions are separated by immunoprecipitation.

17. The method of claim 16, wherein said immunoprecipitation is carried out using a phospho-specific antibody.

18. The method of claim 15, wherein said kinase is a tyrosine kinase.

19. The method of claim 18, wherein said tyrosine kinase is an abl kinase.

20. The method of claim 15, wherein said kinase is a serine or threonine kinase.

21. The method of claim 15, wherein the nucleic acids used to generate said population of nucleic acid-protein fusions of step (a) are cellular mRNA.

22. The method of claim 15, wherein the nucleic acids used to generate said population of nucleic acid-protein fusions of step (a) are synthetic oligonucleotides or nucleic acid fragments.

23. A method for identifying on a nucleic acid microarray a coding sequence for a protein phosphorylated by a kinase comprising the method of claim 15, followed by the further steps comprising:
(e) detectably labeling cDNA complementary to the nucleic acid portions of said enriched population of nucleic acid-protein fusions of step (d);
(f) hybridizing the detectably labeled nucleic acids to a nucleic acid microarray; and
(g) comparing the intensity of label associated with one or more microarray species to the intensity of label associated with said one or more microarray species when alternatively hybridized to labeled cDNA complementary to the nucleic acid portions of the unenriched library of step (a), whereby increased label in association with a microarray species hybridized to said cDNA from said enriched population of nucleic acid-protein fusions compared to the label when hybridized to cDNA from said unenriched library identifies that species as a nucleic acid that encodes a protein phosphorylated by said kinase.

24. A method for identifying a kinase inhibitor, said method comprising:
(a) contacting a population of nucleic acid-protein fusions with a kinase under conditions that allow said nucleic acid-protein fusions to bind to said kinase;
(b) separating kinase-bound nucleic acid-protein fusions from free nucleic acid-protein fusions;
(c) amplifying the nucleic acid portions of the bound nucleic acid-protein fusions;
(d) repeating steps (a)-(c) one or more times, using the amplified nucleic acids of step (c) to generate an enriched population of nucleic acid-protein fusions for use in step (a), thereby identifying one or more nucleic acid-protein fusions that bind said kinase;
(e) separately contacting one or more kinase-binding nucleic acid-protein fusions, or the protein portions thereof, identified in step (d) with said kinase and its substrate under conditions that allow phosphorylation of the substrate by the kinase; and
(f) determining the phosphorylation level of said substrate, a decreased level of phosphorylation of said substrate in the presence of the kinase-binding nucleic acid-protein fusion, or protein portion thereof, relative to the phosphorylation level of said substrate in the absence of the kinase-binding nucleic acid-protein fusion, or protein portion thereof, identifying the protein portion of the kinase-binding nucleic acid-protein fusion as a kinase inhibitor.

25. The method of claim 24, wherein the nucleic acid used to generate said population of nucleic acid-protein fusions of step (a) is cellular mRNA.

26. The method of claim 24, wherein said kinase is a tyrosine kinase.

27. The method of claim 24, wherein said kinase is a serine or threonine kinase.
